SEQUENCE LISTING (41 sequences)

SEQ ID NO: 1 - DNA, 1099 bp, Escherichia coli
  Escherichia coli strain K-12 substrain MG1655 release factor 2 (RF2) cDNA
atgtttgaaattaatccggtaaataatcgcattcaggacctcacggaacgctccgacgttcttagggggtatctttgactacgacgccaagaaagagcgtctggaagaagtaaacgccgagctggaacagccggatgtctggaacgaacccgaacgcgcacaggcgctgggtaaagagcgttcctccctcgaagccgttgtcgacaccctcgaccaaatgaaacaggggctggaagatgtttctggtctgctggaactggctgtagaagctgacgacgaagaaacctttaacgaagccgttgctgaactcgacgccctggaagaaaaactggcgcagcttgagttccgccgtatgttctctggcgaatatgacagcgccgactgctacctcgatattcaggcggggtctggcggtacggaagcacaggactgggcgagcatgcttgagcgtatgtatctgcgctgggcagaatcgcgtggtttcaaaactgaaatcatcgaagagtcggaaggtgaagtggcgggtattaaatccgtgacgatcaaaatctccggcgattacgcttacggctggctgcgtacagaaaccggcgttcaccgcctggtgcgtaaaagcccgtttgactccggcggtcgtcgccacacgtcgttcagctccgcgtttgtttatccggaagttgatgatgatattgatatcgaaatcaacccggcggatctgcgcattgacgtttatcgcacgtccggcgcgggcggtcagcacgttaaccgtaccgaatctgcggtgcgtattacccacatcccgaccgggatcgtgacccagtgccagaacgaccgttcccagcacaagaacaaagatcaggccatgaagcagatgaaagcgaagctttatgaactggagatgcagaagaaaaatgccgagaaacaggcgatggaagataacaaatccgacatcggctggggcagccagattcgttcttatgtccttgatgactcccgcattaaagatctgcgcaccggggtagaaacccgcaacacgcaggccgtgctggacggcagcctggatcaatttatcgaagcaagtttgaaagcagggttatga

SEQ ID NO: 2 - protein, 365 aa, Escherichia coli
  Escherichia coli strain K-12 substrain MG1655 release factor 2 (RF2)
MFEINPVNNRIQDLTERSDVLRGYLDYDAKKERLEEVNAELEQPDVWNEPERAQALGKERSSLEAVVDTLDQMKQGLEDVSGLLELAVEADDEETFNEAVAELDALEEKLAQLEFRRMFSGEYDSADCYLDIQAGSGGTEAQDWASMLERMYLRWAESRGFKTEIIEESEGEVAGIKSVTIKISGDYAYGWLRTETGVHRLVRKSPFDSGGRRHTSFSSAFVYPEVDDDIDIEINPADLRIDVYRTSGAGGQHVNRTESAVRITHIPTGIVTQCQNDRSQHKNKDQAMKQMKAKLYELEMQKKNAEKQAMEDNKSDIGWGSQIRSYVLDDSRIKDLRTGVETRNTQAVLDGSLDQFIEASLKAGL

SEQ ID NO: 3 - protein, 22 aa, Artificial Sequence
  synthetic EGFP peptide containing pActF at UAG site position 182
ADHXQQNTPIGDGPVLLPDNHY

SEQ ID NO: 4 - protein, 8 aa, Artificial Sequence
  synthetic N-terminal FLAG-tag
DYKDDDDK

SEQ ID NO: 5 - protein, 6 aa, Artificial Sequence
  synthetic N-terminal His6 tag, Hisx6, hexahistidine
HHHHHH

SEQ ID NO: 6 - RNA, 25 nt, Artificial Sequence
  synthetic Escherichia coli strain K-12 prfB gene (release factor 2 (RF2)) nucleic acid sequence
cuuaggggguaucuuugacuacgac

SEQ ID NO: 7 - protein, 8 aa, Artificial Sequence
  synthetic Escherichia coli strain K-12 prfB or f-prfB gene (release factor 2 (RF2)) polypeptide sequence
LRGYLDYD

SEQ ID NO: 8 - RNA, 24 nt, Artificial Sequence
  synthetic Escherichia coli strain K-12 f-prfB gene (release factor 2 (RF2)) nucleic acid sequence
cuccgcggcuaucuugacuacgac

SEQ ID NO: 9 - protein, 11 aa, Artificial Sequence
  synthetic sufA gene polypeptide
MDMDYKDDDDK

SEQ ID NO: 10 - protein, 4 aa, Artificial Sequence
  synthetic sufA gene polypeptide
SFGV

SEQ ID NO: 11 - protein, 22 aa, Artificial Sequence
  synthetic sufA gene polypeptide
AVLCLVILKQTLTMSKPGPAAR

SEQ ID NO: 12 - protein, 4 aa, Artificial Sequence
  synthetic yfiA gene polypeptide
VEEE

SEQ ID NO: 13 - protein, 19 aa, Artificial Sequence
  synthetic yfiA gene polypeptide
SFILVSVPTRLRARFLLTA

SEQ ID NO: 14 - RNA, 39 nt, Artificial Sequence
  synthetic RNA transcrription terminator hairpin
ccaacgcgccuucgggcgcguuuuuuguugacagcguga

SEQ ID NO: 15 - protein, 14 aa, Artificial Sequence
  synthetic sufA C-terminal peptide
KAQNECGCGESFGV

SEQ ID NO: 16 - protein, 15 aa, Artificial Sequence
  synthetic sufA C-terminal peptide
KAQNECGCGESFGVY

SEQ ID NO: 17 - protein, 18 aa, Artificial Sequence
  synthetic sufA C-terminal peptide
KQQNECGCGESFGVYAVL

SEQ ID NO: 18 - protein, 10 aa, Artificial Sequence
  synthetic sufA C-terminal peptide
YAVLCLVILK

SEQ ID NO: 19 - protein, 8 aa, Artificial Sequence
  synthetic sufA C-terminal peptide
VLCLCILK

SEQ ID NO: 20 - protein, 7 aa, Artificial Sequence
  synthetic sufA C-terminal peptide
KQLTMSK

SEQ ID NO: 21 - protein, 11 aa, Artificial Sequence
  synthetic yfiA peptide
KDANFVEEVEE

SEQ ID NO: 22 - protein, 12 aa, Artificial Sequence
  synthetic yfiA peptide
KDANFVEEVEEE

SEQ ID NO: 23 - protein, 13 aa, Artificial Sequence
  synthetic yfiA peptide
KDANFVEEVEEEY

SEQ ID NO: 24 - protein, 14 aa, Artificial Sequence
  synthetic yfiA peptide
KDANFVEEVEEEYS

SEQ ID NO: 25 - protein, 15 aa, Artificial Sequence
  synthetic yfiA peptide
KDANFVEEVEEEYSF

SEQ ID NO: 26 - protein, 17 aa, Artificial Sequence
  synthetic yfiA peptide
KDANFVEEVEEEYSFIL

SEQ ID NO: 27 - protein, 18 aa, Artificial Sequence
  synthetic yfiA peptide
KDANFVEEVEEEYSFILS

SEQ ID NO: 28 - protein, 21 aa, Artificial Sequence
  synthetic yfiA peptide
KDANFVEEVEEEYSFILSPTR

SEQ ID NO: 29 - DNA, 87 bp, Artificial Sequence
  synthetic human histone H3a wild type (WT) nucleic acid sequence
atgactagtgctcgtaccaaacagaccgctcgtaaatctaccggtggtaaagctccgcgtaaacagctggctaccaaagctgctcgt

SEQ ID NO: 30 - DNA, 87 bp, Artificial Sequence
  synthetic human histone H3a 1-TAG nucleic acid sequence
atgactagtgctcgtaccaaacagaccgctcgttagtctaccggtggtaaagctccgcgtaaacagctggctaccaaagctgctcgt

SEQ ID NO: 31 - DNA, 87 bp, Artificial Sequence
  synthetic human histone H3a 2-TAG nucleic acid sequence
atgactagtgctcgtaccaaacagaccgctcgttagtctaccggtggttaggctccgcgtaaacagctggctaccaaagctgctcgt

SEQ ID NO: 32 - DNA, 87 bp, Artificial Sequence
  synthetic human histone H3a 3-TAG nucleic acid sequence
atgactagtgctcgtaccaaacagaccgctcgttagtctaccggtggttaggctccgcgttagcagctggctaccaaagctgctcgt

SEQ ID NO: 33 - DNA, 87 bp, Artificial Sequence
  synthetic human histone H3a 4-TAG nucleic acid sequence
atgactagtgctcgtaccaaacagaccgctcgttagtctaccggtggttaggctccgcgttagcagctggctacctaggctgctcgt

SEQ ID NO: 34 - protein, 41 aa, Artificial Sequence
  synthetic tryptic fragment of EGFP position 182 peptide
HNIEDGSVQLADHXQQNTPIGDGPVLLPDNHYLSTQSALSK

SEQ ID NO: 35 - protein, 5 aa, Artificial Sequence
  synthetic penta-His
HHHHH

SEQ ID NO: 36 - protein, 15 aa, Artificial Sequence
  synthetic tryptic fragment of EGFP position 39 peptide
FSVSGEGEGDATXGK

SEQ ID NO: 37 - protein, 16 aa, Artificial Sequence
  synthetic tryptic fragment of EGFP position 151 peptide
LEYNYNSHNVXIMADK

SEQ ID NO: 38 - protein, 41 aa, Artificial Sequence; identical to SEQ ID NO: 34
  synthetic tryptic fragment of EGFP position 182 peptide

SEQ ID NO: 39 - protein, 22 aa, Artificial Sequence
  synthetic EGFP peptide containing Gln at UAG site position 182
ADHQQQNTPIGDGPVLLPDNHY

SEQ ID NO: 40 - protein, 29 aa, Artificial Sequence
  synthetic human histone H3a wild type (WT) N-terminal sequence (H3a Seq)
MTSARTKQTARKSTGGKAPRKQLATKAAR

SEQ ID NO: 41 - protein, 9 aa, Artificial Sequence
  synthetic yfiA gene polypeptide
MDYKDDDDK

(19) United States
(12) Patent Application Publication
(10) Pub. No.: US 2014/0011235 A1
(43) Pub. Date: Jan. 9, 2014

(54) RELEASE FACTOR 1 (RF1) IN ESCHERICHIA COLI

(71) Applicant: Lei Wang, La Jolla, CA (US)

(72) Inventor: Lei Wang, La Jolla, CA (US)

(73) Assignee: Salk Institute For Biological Studies, La Jolla, CA (US)

(21) Appl. No.: 13/991,115

(22) PCT Filed: Nov. 29, 2011
(86) PCT No.: PCT/US11/62466

§ 371 Date: Aug. 12, 2013

Related U.S. Application Data
(60) Provisional application No. 61/419,110, filed on Dec. 2, 2010.
(60) Provisional application No. 61/500,055, filed on Jun. 22, 2011.

(51) Int. Cl. C12N 15/70; C12P 21/00

(52) U.S. Cl. CPC C12N 15/70; C12P 21/00
USPC 435/69.1; 435/252.33; 435/471

(57) ABSTRACT

Provided herein are release factor 1 (RF1) deficient bacteria, methods of using the bacteria to reassign the UAG codon, and generate mutant polypeptides.

### CROSS-REFERENCES TO RELATED APPLICATIONS

The present application claims priority to U.S. Ser. No. 61/419,110, filed Dec. 2, 2010, the disclosure of which is incorporated herein in its entirety.

### STATEMENT AS TO RIGHTS TO INVENTIONS MADE UNDER FEDERALLY SPONSORED RESEARCH AND DEVELOPMENT

This invention was made with Government support under Grant No. 1DP2OD004744 awarded by the National Institutes of Health. The Government has certain rights in the invention.

### BACKGROUND OF THE INVENTION

Protein translation is terminated by class I release factors (RFs) (Youngman et al., Annu. Rev. Microbiol., 62, 353-373 (2008)). In prokaryotes, RF1 recognizes stop codon UAA and UAG, while RF2 is specific for UAA and UGA (Scolnick et al., Proc. Natl. Acad. Sci. U.S.A., 61, 768-774 (1968)). In eukaryotes and archaea, however, a single RF decodes all three stop codons (Frolova et al., Nature, 372, 701-703 (1994); Dontsova et al., FEBS Lett., 472, 213-216 (2000)). UAG is used for termination in ˜7% of E. coli genes (Nakamura et al., Nucleic Acids Res. 28, 292 (2000)), and RF1 has been reported to be essential for E. coli (Rydén & Isaksson, Mol. Gen. Genet., 193, 38-45 (1984); Gerdes et al., J. Bacteriol., 185, 5673-5684 (2003)).

Translation termination is a critical step of converting genetic information into proteins. When a stop codon (UAA, UAG or UGA) is in the A site of the ribosome, a class I release factor (RF) instead of an aminoacylated tRNA recognizes the signal and promotes peptide release from the tRNA in the P site. While eukaryotes and archaea use a single class I RF (eRF1 and aRF1, respectively) to recognize all three stop codons, bacteria use RF1 and RF2. RF1 and RF2 are homologous, but they are dissimilar to the eRF1 and aRF1 in sequence and structure (Song et al., Cell, 100, 311-321 (2000); Laurberg et al., Nature, 454, 852-857 (2008); Korostelev et al., EMBO J., 29, 2577-2585 (2010)). In some eukaryotes such as ciliates and green algae, convergent changes in eRF1 have been associated with the reassignment of a stop codon to a sense codon, creating deviations from the standard genetic code (Knight et al., Nat. Rev. Genet., 2, 49-58. (2001)). For instance, the eRF1 of Tetrahymena restricts its recognition to UGA, and UAA/UAG are reassigned to Gln; the eRF1 of Euplotes recognizes UAA/UAG only as stop codons, and UGA is used to encode Cys (Lozupone et al., Curr. Biol., 11, 65-74 (2001); Inagaki & Doolittle, Nucleic Acids Res. 29, 921-927 (2001)). To date no free-living bacterium has been found to lack either RF1 or RF2.

Stop codons have been exploited for the incorporation of both natural and unnatural amino acids into proteins. Occurring only once per gene, the relative scarcity of stop codons mitigates any damage caused by codon reassignment. Natural suppressor tRNAs that read stop codons as common amino acids have been identified in E. coli and other organisms (Benzer, S, and Champe, S. P., Proc. Natl. Acad. Sci. U.S.A. 48, 1114-21 (1962); Beier, H. and Grimm, M., Nucleic Acids Res. 29, 4767-82 (2001)).

Furthermore, orthogonal tRNA/synthetase pairs have been generated to incorporate unnatural amino acids into proteins in response to a stop codon (Wang et al., Science 292, 498-500 (2001); Wang and Schultz, Chem. Int. Ed. Engl. 44, 34-66. (2004)). Using this approach, unnatural amino acids with a variety of functional groups have been genetically incorporated into proteins in both prokaryotic and eukaryotic cells (Wang et al., Chem. Biol. 16, 323-36 (2009)).

The incorporation efficiency of both natural and unnatural amino acids is, however, limited because suppressor tRNAs are in competition with endogenous release factors, the native function of which is to recognize stop codons and terminate translation. Stop codon assignment is therefore ambiguous, limiting the potential of this technology. Release factor competition also results in truncated protein products, which can interfere with target protein function or be deleterious to the host. Low incorporation efficiency also prevents the synthesis of proteins containing unnatural amino acids at multiple sites. Protein yields drop precipitously with the addition of even a second stop codon. It is thus currently infeasible to efficiently synthesize proteins with unnatural amino acid modifications at multiple sites. The present disclosure describes cells, compositions, and methods directed toward reassignment of the stop codon that address these problems.

### BRIEF SUMMARY OF THE INVENTION

In some embodiments, the invention provides release factor 1 (RF1) deficient bacterial cells that are viable (able to grow). In some embodiments, the bacteria lack the full length coding sequence of RF1, e.g., are RF1 knockout. In some embodiments, the bacteria are recombinant. In some embodiments, the bacteria are Escherichia coli (E. coli). In some embodiments, the E. coli are from a parental strain selected from the group consisting of REL606, BL21, BL21 (DE3), and DH10βf. In some embodiments, the bacteria are a species from a bacterial genera selected from the group consisting of Salmonella, Anaplasma, Butyrivibrio, Rhodococcus, Bifidobacterium, Laribacter, Pantoea, Mycobacterium, Glossina, Helicobacter, Synechococcus, Synechocystis, Caulobacter, Streptomyces, Rickettsia, Campylobacter, Neisseria, Arcobacter, Streptococcus, Staphylococcus, Yersinia, Bordetella, Candidatus, Chlamydia, and Borrelia.

In some embodiments, the bacteria comprise a functional RF2 protein, e.g., having RF2 activity that is at least 20, 30, 40, 50, 60, 70, 80, 90, or 100% of the RF2 in a B strain E. coli, or higher. The RF2 activity can be, e.g., interaction with a UAA stop codon and/or termination of protein translation. In some embodiments, the RF2 is endogenous. In some embodiments, the RF2 protein has an alanine at the amino acid position corresponding to 246 of SEQ ID NO:2. In some embodiments, the RF2 protein has a glutamic acid at the amino acid position corresponding to 293 of SEQ ID NO:2. In some embodiments, the RF2 protein is recombinantly introduced into the bacterial cell. For example, the bacteria can include a recombinant nucleic acid that encodes for a functional RF2 protein.

In some embodiments, the UAG codon is recognized by an aminoacylated tRNA in the bacteria, and results in incorporation of an amino acid into a nascent protein strand. That is, the meaning of the UAG stop codon is reassigned in the bacteria so that it encoded for an amino acid. In some embodiments, the tRNA is endogenous. In some embodiments, the amino acid is selected from the group consisting of tyrosine, glutamine, and tryptophan. In some embodiments, the tRNA is exogenous. In some embodiments, the amino acid is a non-naturally occurring amino acid. In some embodiments, the amino acid is a naturally occurring amino acid.

In some embodiments, the RF1 deficient bacteria grow at a similar rate to the parental strain (i.e., the corresponding RF1 wild type strain). In some embodiments, the RF1 deficient bacteria grow at a slower rate, e.g., 10, 20, 30, 40, 50, 60, 70, 80, or 90% slower, or a 1.5, 2, 3, 4, 5, 6, 7, 8, 9, 10, or 20-fold slower rate than the parental strain. Growth can be described as is customary in the art, e.g., in doubling times.

In some embodiments, the RF1 deficient bacteria comprise a recombinant nucleic acid (e.g., an exogenous nucleic acid) encoding a protein comprising a mutant amino acid, wherein said mutant amino acid is encoded by a TAG codon where the recombinant nucleic acid is DNA, or by a UAG codon where the recombinant nucleic acid is RNA. In some embodiments, the mutant nucleic acid is a non-naturally occurring amino acid. In some embodiments, the amino acid is a naturally occurring amino acid that is not found in the wild type form of the protein. In some embodiments, the protein comprises more than one mutant amino acid, e.g., 2, 3, 4, 5, 6, 7, 8, 2-10, 5-10, or more than 10 mutant amino acids. The multiple mutant amino acids can be different or the same in any combination (e.g., ½ a first mutant amino acid, and ½ a second mutant amino acid).

In some embodiments, the RF1 deficient bacteria comprise a recombinant nucleic acid (e.g., an exogenous nucleic acid) encoding an orthogonal tRNA comprising a CUA anticodon; and a recombinant nucleic acid encoding an orthogonal synthetase capable of functionally binding to said orthogonal tRNA. In some embodiments the orthogonal tRNA and synthetase are encoded on the same nucleic acid (e.g., on the same expression vector). In some embodiments, the RF1 bacteria include a first recombinant nucleic acid encoding a protein comprising the mutant amino described above, a second recombinant nucleic acid encoding an orthogonal tRNA comprising a CUA anticodon; and a third recombinant nucleic acid encoding an orthogonal synthetase capable of functionally binding to said orthogonal tRNA. In some embodiments, all three coding sequences are included on the same nucleic acid (e.g., on the same expression vector).

RF1 deficient bacteria comprising the three coding sequences described above can be used in methods for generating (producing) the protein comprising at least one mutant amino acid. In some embodiments, the method for producing a protein comprising at least one amino acid is practiced on a large scale, e.g., to produce gram or kilogram quantities of protein.

In some embodiments, the invention provides methods for reassigning the UAG codon in a bacterial cell, comprising rendering the bacterial cell RF1 deficient. In some embodiments, the rendering comprises recombinant disruption of the endogenous RF1 gene in the bacterial cell. In some embodiments, the method comprises deleting at least part of the RF1 gene in the bacterial cell. In some embodiments, the method further comprises transfecting into the bacterial cell (i.e., introducing into the bacterial cell) a recombinant nucleic acid (e.g., an exogenous nucleic acid) encoding a protein comprising a mutant amino acid, wherein said mutant amino acid is encoded by a TAG codon where the recombinant nucleic acid is DNA, or by a UAG codon where the recombinant nucleic acid is RNA. In some embodiments, the mutant nucleic acid is a non-naturally occurring amino acid. In some embodiments, the amino acid is a naturally occurring amino acid that is not found in the wild type form of the protein.

In some embodiments, the bacteria are E. coli. In some embodiments, the E. coli are from a parental strain selected from group consisting of REL606, BL21, BL21 (DE3), and DH10βf. In some embodiments, the bacteria are a species from a bacterial genera selected from the group consisting of Salmonella, Anaplasma, Butyrivibrio, Rhodococcus, Bifidobacterium, Laribacter, Pantoea, Mycobacterium, Glossina, Helicobacter, Synechococcus, Synechocystis, Caulobacter, Streptomyces, Rickettsia, Campylobacter, Neisseria, Arcobacter, Streptococcus, Staphylococcus, Yersinia, Bordetella, Candidatus, Chlamydia, and Borrelia.

In some embodiments, the bacteria comprise a functional RF2 protein, e.g., having RF2 activity that is at least 20, 30, 40, 50, 60, 70, 80, 90, or 100% of the RF2 in a B strain E. coli, or higher. In some embodiments, the RF2 protein has an alanine at the amino acid position corresponding to 246 of SEQ ID NO:2. In some embodiments, the RF2 protein has a glutamic acid at the amino acid position corresponding to 293 of SEQ ID NO:2. In some embodiments, the RF2 is endogenous. In some embodiments, the method further comprises introducing a functional RF2 protein into the bacterial cell. For example, the method can further comprise transfecting the bacteria with a recombinant nucleic acid that encodes for a functional RF2 protein.

Further provided are kits for practicing the methods of the invention. In some embodiments, the kit comprises RF1 deficient bacteria as described herein, e.g., in a container. In some embodiments, the bacteria are frozen or lyophilized. In some embodiments, the kit further comprises a recombinant nucleic acid encoding an orthogonal tRNA comprising a CUA anticodon; and a recombinant nucleic acid encoding an orthogonal synthetase capable of functionally binding to said orthogonal tRNA. In some embodiments, the two coding sequences are included on the same nucleic acid (e.g., expression vector or plasmid). In some embodiments, the kit further comprises an expression vector for expressing a recombinant nucleic acid encoding a protein with at least one mutant amino acid, wherein the mutant amino acid is encoded by a UAG or TAG codon, for RNA or DNA respectively. In some embodiments, the expression vector or plasmid comprising the orthogonal tRNA and synthetase encoding sequences includes a site for adding the nucleic acid encoding the protein with at least one mutant amino acid. In some embodiments, the kit further comprises a recombinant nucleic acid encoding a functional RF2. The nucleic acid encoding the RF2 protein can also be included on the same nucleic acid as the orthogonal tRNA and synthetase encoding sequences, and/or the mutant protein encoding sequence. In some embodiments, the kit further comprises at least one amino acid for use as the mutant amino acid.

### BRIEF DESCRIPTION OF THE DRAWINGS

FIG. 1. RF1 can be knocked out in E. coli strains containing a wild type RF2. A. The RF1-encoding gene prfA was replaced with the chloramphenicol acetyltransferase (cat) gene through homologous recombination using the phage λ red recombinase expressed with plasmid pKD46. Chloramphenicol resistant colonies were screened using PCR for cat replacement and confirmed with sequencing. The agarose gels show the PCR results for parental and knockout strains as indicated. B. A list of E. coli strains tested for RF1 knockout. K-12 and B strains are the most widely used E. coli and subjects of classical experiments. RF1 was successfully knocked out in three B strains and the DH10βf strain.

FIG. 2. The UAG codon in RF1 knockout strain JX1.0 is misread by endogenous tRNAs. A. An EGFP gene with UAG codon at permissive sites was driven by the arabinose promoter in plasmid pBAD-EGFP(n-TAG) to test EGFP expression. B. In-cell fluorescence intensity measured with fluorometry for BL21(DE3) and JX1.0 cells transformed with pBAD-EGFP(1-TAG) or pBAD-EGFP(3-TAG). C. High resolution mass spectrometric analysis of EGFP protein purified from JX1.0 expressing pBAD-EGFP(1-TAG). The spectrum shows the intact precursor ions (charge deconvoluted) for the tryptic fragment of EGFP (HNIEDGSVQLADHXQQNTP IGDGPVLLPDNHYLSTQSALSK, X representing the UAG encoded site). Monoisotopic masses indicate that Gln, Tyr, and Trp (expected [M+H]⁺=4437.17, 4472.17, and 4495.19 Da, and measured 4437.16, 4472.16, and 4494.16 Da, respectively) were incorporated at the UAG site. No other amino acids were detected at the UAG site.

FIG. 3. Use of an orthogonal tRNA/synthetase pair to reassign the UAG codon to an amino acid in JX1.0. A. The All-in-One plasmid pAIO-EGFP(n-TAG) expresses the orthogonal amber suppressor tRNA_{CUA}^{Tyr} which specifically recognizes the UAG codon, the orthogonal synthetase LW1RS which is specific for the unnatural amino acid pActF, and EGFP reporter gene with different number of UAG codons at permissive sites. Structure of pActF is shown to the right. B. Western blot analysis of EGFP expression by pAIO-EGFP(n-TAG) in BL21(DE3) and JX1.0 cells in the presence or absence of pActF. A penta-His antibody was used to detect the His×6 tag fused at the N-terminus of EGFP. C. In-cell fluorescence intensity of EGFP expressed by pAIO-EGFP(n-TAG) in BL21(DE3) and JX1.0 cells in the presence or absence of pActF. Samples were normalized for constant cell numbers for each lane. Measurement was performed on 3 independent batches of cells and error bars represent s.e.m. D. SDS-PAGE analysis of EGFP proteins purified from JX1.0 cells expressing pAIO-EGFP (n-TAG) in the absence of presence of pActF. The commassie blue stained gel show a high level of protein purity. E. High resolution mass spectrometric analysis of EGFP expressed by pAIO-EGFP(3-TAG) in JX1.0 with pActF in the growth media. The spectra show the intact precursor ions (charge deconvoluted) for the tryptic fragments of EGFP. Monoisotopic masses indicate that pActF was incorporated in response to the UAG codon at all three UAG sites. Top: position 39, peptide FSVSGEGEGDATXGK, X representing the UAG encoded site; expected [M+H]⁺=1529.65 Da, measured 1529.66 Da. Middle: position 151, peptide LEYNYNSHNVXIMADK, expected [M+H]⁺=1999.89 Da, measured 1999.90 Da; the 2015.89 peak corresponds to Met oxidation of this pActF-containing peptide. Bottom: position 182, peptide HNIEDGSVQLADHXQQNTPIGDGPVLLPDNHY LSTQSALSK, expected [M+H]⁺=4498.18 Da, measured 4498.14 Da. No peaks corresponding to any other amino acids at these UAG positions were detected. The signal-to-noise ratio is >1000, translating to a fidelity for pActF incorporation of >99.9%. Tandem MS spectra of the peptides are shown in FIG. 4, which unambiguously confirm the presence of pActF at position X. The results indicate that misreading of UAG by near-cognate tRNAs was outcompeted by specific pActF incorporation.

FIG. 4. Mass spectrometry of pActF EGFP A. Tandem mass spectrum of peptide FSVSGEGEGDATXGK, with X representing the UAG encoded site at position 39. The sequence of the peptide containing ActF at position X was assigned from the annotated b and y ion series. B. Tandem mass spectrum of peptide LEYNYNSHNVXIMADK, X representing the UAG encoded site at position 151. The sequence of the peptide containing ActF at position X was assigned from the annotated b and y ion series. C. Tandem mass spectrum of peptide HNIEDGSVQLADHXQQNTPIGDGPVLLPDNHYLSTQSALSK, X representing the UAG encoded site at position 182. The sequence of the peptide containing ActF at position X was assigned from the annotated b and y ion series.

FIG. 5. JX1.0 with RF1 knockout and UAG reassignment are viable. The growth curves for the RF1 knockout strain JX1.0 are represented with closed symbols and for the parental BL21 (DE3) strain with open symbols. Squares represent cells only; circles represent cells transformed with the pAIO plasmid, which expresses the orthogonal tRNA_{CUA}^{Tyr}/LW1RS pair; and triangles represent cells transformed with the pAIO plasmid and grown in the presence of pActF. Error bars represent s.e.m., n=3.

FIG. 6. RF1 can be knocked out in a genome-reduced, functional RF2 E. coli strain. A. Features of the prfB gene encoding the RF2 in K-12 E. coli strains: an in-frame UGA stop codon for autoregulation of RF2 expression and the Ala246Thr mutation impairing RF2 recognition of the UAA codon. pfrB nucleic acid sequence depicted: SEQ ID NO:6; pfrB, pfrB^{f} polypeptide depicted: SEQ ID NO:7. In prfB^{f}, UGA regulation was removed and residue 246 was reverted to Ala. A Shine-Dalgarno like sequence in prfB was silently mutated to a Sac II site in prfB^{f} to facilitate the screening of prfB^{f} knock-in. prfB^{f} nucleic acid depicted: SEQ ID NO:8. B. Generation of the JX2.0 strain. The prfB gene in MDS42 was first replaced with prfB^{f} followed by a Cm resistant cat cassette. The cat cassette was subsequently removed. C. Generation of the JX3.0 strain. The prfA gene encoding RF1 was successfully knocked out with a cat cassette in JX2.0 to afford JX3.0. D. Illustration of main features of the three strains, MDS42, JX2.0 and JX3.0. E. Growth rates of the JX2.0 and JX3.0 strains with or without the tyrosyl suppression system (pAIO-TyrRS, see FIG. 7). Colony JX31 represents the JX3.0 strain. OD₆₀₀ values were recorded at different time points for each strain. Growth rates of JX2.0 (□) and JX33 (Δ) were assayed in the absence (filled) or presence (open) of the pAIO-TyrRS plasmid. Only JX33+pAIO-TyrRS showed a slowing of growth. All strains still reached saturation within 10 hours of growth. Shown is the average from three independent measurements with error bars representing s.e.m.

FIG. 7. Removal of RF1 enables encoding natural or unnatural amino acids at multiple UAG sites in JX33. A. The All-in-One plasmid was used to express the orthogonal amber suppressor tRNA with a CUA anticodon, an orthogonal synthetase, and the EGFP reporter with UAG codons. Structures of Tyr and pActF are shown to the right. B. Western analysis of EGFP expression when UAG codons were decoded as Tyr by pAIO-TyrRS. pAIO-TyrRS with the EGFP gene containing 1, 2, 3, or 6 UAG codons was transformed into JX2.0 and JX33. The same number of cells for each sample were lysed, separated on SDS-PAGE, and probed with a penta-His antibody. C. In-cell fluorescence assay of EGFP intensity when UAG codons were decoded as Tyr by pAIO-TyrRS. The same number of cells was used for each sample. Measurement was performed on 3 independent batches of cells and error bars represent s.e.m. JX2.0 had decreased expression of EGFP with each addition of UAG, and the 6-TAG construct showed no expression of EGFP. D. Western analysis of EGFP expression when UAG codons were decoded as the unnatural amino acid pActF by pAIO-LW1RS. Conditions are the same as in FIG. 7B. For each sample, a duplicate of cultures were grown in the presence or absence of pActF in the growth media. E. In-cell fluorescence assay of EGFP intensity when UAG codons were decoded as pActF by pAIO-LW1RS. Measurements were performed as in FIG. 7C with duplicate samples grown in the presence or absence of pActF in the growth media. n=3, error bars represent s.e.m. With UAG decoded as pActF, JX2.0 produced full-length EGFP in only the 1-TAG EGFP construct, while JX33 produced similar amounts of EGFP in the 1-, 2- and 3-TAG constructs, with EGFP detectable in the 6-TAG construct. F. Fluorescence images of cells when UAG was decoded as Tyr (center) or pActF (right).

FIG. 8. pActF is selectively incorporated at multiple UAG sites with high fidelity in JX33. A. MS/MS spectrum of EGFP peptide ADHUQQNTPIGDGPVLLPDNHY (SEQ ID NO:3). U represents the UAG codon at residue 182. Star (*) in the spectrum denotes peptide fragments containing pActF, which unambiguously indicate that pActF was incorporated at the UAG site. B. Extracted ion chromatograms (EIC) of the above peptide containing pActF (top) or Gln (bottom) at the UAG 182 position. The peak areas are indicated. C. Summary of all UAG site-containing peptides from the 1-, 2-, and 3-TAG EGFP mutants. Peptide intensities were determined by the area peak in the EIC and translated into the incorporation fidelity for pActF.

FIG. 9. Ten UAG sites can be simultaneously suppressed with natural or unnatural amino acids in JX33. A. GFP structure (Protein data base entry: 1GFL) illustrating the sites where UAG was introduced. In the 10-TAG reporter, codons for 10 residues highlighted were mutated to UAG. In the 10-TAGtd reporter, 10 consecutive UAG codons were inserted to replace E172 and D173. B. Western blot analysis of the expression of 10-TAG and 10-TAGtd EGFP in JX33. The UAG codons were decoded as Tyr by tRNA_{CUA}^{Tyr}/TyrRS or as pActF by tRNA_{CUA}^{Tyr}/LW1RS. Cell lysates from same amount of cells were separated by SDS-PAGE and probe with a penta-His antibody.

FIG. 10. Various unnatural amino acids can be incorporated at multiple UAG sites in JX33. A. Western analysis of the expression of 3-TAG EGFP reporter in JX33 with the UAG codon decoded as different unnatural amino acids (Uaa) by different orthogonal tRNA/synthetase pairs. ActK was incorporated by an orthogonal pair derived from tRNA^{Pyl}/PylRS, and the other 3 were by pairs derived from tRNA^{Tyr}/TyrRS. The same number of cells were used for each sample. After cell lysis, proteins were separated on SDS-PAGE and probed with a penta-His antibody. Densitometric analysis of Western blot bands and purified protein yields (Table 2) were consistent on incorporation efficiency. B. In-cell fluorescence assay of the mutant EGFP proteins containing different unnatural amino acids at 3 UAG sites. Measurements were performed using 3 independent batches of cells. Error bars represent s.e.m. C. The N-terminal sequence of human histone H3a and UAG codons introduced at the known acetylation sites. Sequences are depicted as follows: WT=SEQ ID NO:29; 1-TAG=SEQ ID NO:30; 2-TAG=SEQ ID NO:31; 3-TAG=SEQ ID NO:32; 4-TAG=SEQ ID NO:33. D. Western analysis showing that full-length histone H3a was expressed for 1-, 2-, 3-, and 4-TAG H3a constructs in the presence of pActF or ActK in JX33 cells. For pActF, the sample loading ratio was 1:1:3:3; for ActK, the sample loading ratio was 1:3:7:9. E. Yields of H3a proteins were calculated after purification. F. SDS-PAGE analysis of GST proteins expressed in JX33 with pActF incorporated at 1, 2, and 3 UAG sites. GST was purified with Ni-NTA chromatography, separated on the gel and stained with Coomassie blue. Loading was normalized to the same number of cells. GST yields were 67 (±11), 57 (±12), and 68 (±9) mg/L for 1-, 2-, and 3-TAG mutants, respectively. Measurement was performed on 3 independent batches of cells and errors represent s.e.m.

FIG. 11. Endogenous genes ending in TAG extend their peptides to different positions in an mRNA context-dependent manner in JX33. A. Two categories of endogenous genes ending in TAG: sufA representing those with an in-frame, non-UAG stop codon downstream before a transcription terminator (shown as a hairpin); yfiA representing those with a terminator before the next in-frame, non-UAG stop codon. A FLAG-tag (DYKDDDDK) (SEQ ID NO:4) was inserted scarlessly to the N-terminus of these two proteins in the genome to facilitate protein detection. Polypeptides depicted: MDMDYKDDDK (SEQ ID NO:9); SFGV (SEQ ID NO:10); AVLCLVILKQTLTMSKPGPAAR (SEQ ID NO:11); VEEE (SEQ ID NO:12); SFILSVSPTRLRARFLLTA (SEQ ID NO:13). RNA depicted: CCAACGCGCCUUCGGGCGCGUUUUUUGUUGACAGCGUGA (SEQ ID NO:14). B. Western analysis of SufA purified from cells using the N-terminal FLAG tag. Extension of SufA to its next stop codon occurred only in the JX33 strain harboring the suppressor tRNA_{CUA}^{Tyr}/TyrRS (lanes 4 and 6). All other strains showed a band that corresponds to the wild type SufA molecular weight. Loading was normalized for cell numbers in lane 1-4. Lane 5 and 6 were duplicates of lane 2 and 4, respectively, with increasing amounts to clearly show the SufA protein extension in JX33 but not in JX2.0. An anti-FLAG antibody was used for detection. Polypeptide depicted: SEQ ID NOs:15-20. C. Tandem MS analysis of SufA protein purified from JX3.0 harboring tRNA_{CUA}^{Tyr}/TyrRS confirmed that the protein was extended to the next in-frame UGA codon and a Tyr was incorporated at the UAG site. A small amount of non-extended wild type SufA C-terminal peptide (bolded) was also detected by the more sensitive MS but not on Western. D. Western analysis of YfiA purified from cells using the N-terminal FLAG tag. Loading in lane 1, 2, 3 and 5 were normalized to the same amount of cells. Loading in lane 4 is 1/50 of lane 3, which showed it is a single band. Lane 6 is a re-run of lane 5 sample in a higher percentage gel (20% vs. 15%) for longer time to achieve better separation. YfiA in JX3.0 harboring tRNA_{CUA}^{Tyr}/TyrRS (lane 5 and 6) showed 3 predominant bands, while other samples showed only one band. E. Tandem MS analysis of YfiA purified from JX33 harboring tRNA_{CUA}^{Tyr}/TyrRS showed that no peptide was extended to the next UGA codon. Various extensions before the terminator hairpin structure were identified, suggesting an early ribosome drop-off. Peptides extended to the 0, 2nd and 6th amino acid after UAG were predominant and underlined. Polypeptides depicted: SEQ ID NOs:21-28.

FIG. 12. Full genome sequencing of JX2.0, JX31, and JX33 confirms knockout of RF1. A. Deletion of prfA in JX31 and JX33 was confirmed. B. The A293E mutation cannot rescue the RF1 temperature sensitive phenotype of the MRA8 strain. We replaced the prfB gene in the genome of the tsRF1 strain MRA8 with the prfB^{f} (A293E) gene from JX33, generating the MR8 A293E strain. Cells were grown at both 37° C. and 43° C. to assay for any change in growth defects. The introduction of prfB^{f} (A293E) did not rescue the growth defect of MRA8 at 43° C. The control strain DH10β had no such defect.

FIG. 13. UAA incorporation at UAG sites is dramatically increased in JX33 compared to BL21 expressing L11C. SDS-PAGE analysis of EGFP protein with Uaa pActF incorporated at 1-UAG and 3-UAG sites in JX33 or in BL21(DE3) with L11C (C terminus of ribosomal protein L11). The same pAIO plasmid containing the pActF-specific tRNA_{CUA}^{Tyr}/LW1RS pair and EGFP(TAG) mutant gene was used for expression in both JX33 and BL21 cells. L11C was coexpressed in BL21 cells using the pET-L11C plasmid as described (Huang et al. (2010) Mol. Biosyst. 6:686). EGFP was purified with Ni-NTA chromatography, separated on the gel and stained with Coomassie blue. Loading was normalized to the same number of cells. EGFP yields were determined after Ni-NTA purification.

### DETAILED DESCRIPTION OF THE INVENTION

### I. Introduction

The present disclosure shows the feasibility of codon reassignment in bacteria, thereby providing a model system to investigate the cellular adaptions of code evolution. In prokaryotes, stop codons are recognized by two release factors, RF1 for UAA/UAG and RF2 for UAA/UGA (Scolnick et al., Proc. Natl. Acad. Sci. U.S.A. 61, 768-74 (1968)). To achieve full reassignment of the amber codon UAG, the inventors removed RF1 from the system. The prfA gene encoding RF1 has been reported as essential for E. coli survival (Rydén and Isaksson, Mol. Gen. Genet. 193, 38-45 (1984); Gerdes et al., J. Bacteriol. 185, 5673-84 (2003)). The present results show that RF1 gene expression or activity can be eliminated in E. coli genome in the presence of functional RF2 (e.g., with an alanine at a position corresponding to amino acid 246 of SEQ ID NO:2). The RF1 knockout strain is viable, stable, and sustainable. RF1 deficient E. coli allows for the genetic incorporation of a variety of natural and unnatural amino acids into proteins at the UAG site, as deficiency of RF1 essentially reassigns UAG to a sense codon. The data show that UAG codons terminating endogenous genes are also suppressed, but do not have a negative effect on overall cell fitness. Through whole genome sequencing the inventors identified a novel mutation in RF2, which can also contribute to the unique phenotype of the JX3.0 E. coli strain.

### II. Definitions

Unless defined otherwise, technical and scientific terms used herein have the same meaning as commonly understood by a person of ordinary skill in the art. See, e.g., Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989); Tijssen, TECHNIQUES IN BIOCHEMISTRY AND MOLECULAR BIOLOGY (1993). Any methods, devices and materials similar or equivalent to those described herein can be used in the practice of this invention. The following definitions are provided to facilitate understanding of certain terms used frequently herein and are not meant to limit the scope of the present disclosure.

A cell is considered “deficient” for a given factor if the activity or expression of the factor is significantly reduced, e.g., reduced by at least 10, 20, 30, 40, 50, 60, 70, 80, 90, 95, 98, or 100% compared to a non-deficient or normal cell. A cell or organism can be rendered deficient for a factor by genetic manipulation (e.g., knock out or knock down) or use of antisense or siRNA to reduce expression. Alternatively, the activity of the factor can be reduced using an antagonist or inhibitor, e.g., to interfere with binding or other activities.

A cell is considered “viable” if it is alive and capable of growth. The number of viable bacterial cells in a given sample can be determined directly, e.g., using a microscope, or using plate counts at various dilutions. Roszak and Colwell (1987) Applied Environ. Microbiol. 53:2889 describe an additional method for determining viability based on incorporation of radiolabeled substrates.

The terms “nucleic acid,” “oligonucleotide,” “polynucleotide,” and like terms typically refer to polymers of deoxyribonucleotides or ribonucleotides in either single- or double-stranded form, and complements thereof. The term “nucleotide” typically refers to a monomer. The terms encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

Transfer RNA or “tRNA” represents a subset of RNA molecules that can recognize a codon on mRNA using a complementary anticodon, and transfer the amino acid that corresponds to the recognized codon to the nascent (growing) protein strand. This is not the case for stop codons, as described in more detail herein. The proper amino acid is loaded on to the tRNA by aminoacyl synthetase. An “aminoacylated tRNA” refers to the tRNA loaded with its corresponding amino acid. “Orthogonal” tRNA and synthetase pairs can be introduced into a cell from a different strain or species to change the meaning of a given codon within the cell (see, e.g., Xie and Schultz (2005) Curr. Opin. Chem. Biol. 9:548; Wang et al. (2000) J. Am. Chem. Soc. 122:5010).

A “release factor” refers to a protein that allows for termination of protein translation by recognizing stop codons. As described above, most codons encode for a particular amino acid, which is provided by a tRNA. Stop codons, however, are recognized by a release factor, which are described in more detail herein. Prokaryotes have RF1 (recognizing UAA and UAG) and RF2 (recognizing UAA and UGA). Eukaryotes typically rely on a single release factor, eRF1 (Weaver (2005). Molecular Biology, p. 616-621. McGraw-Hill, New York, N.Y.).

The term “gene” refers to a segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene (e.g., promoters, enhancers, etc.). A “gene product” can refer to either the mRNA or protein expressed from a particular gene.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “transfection” or “transfected” refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

An expression vector refers to a nucleic acid that includes a coding sequence and sequences necessary for expression of the coding sequence. The expression vector can be viral or non-viral. A “plasmid” is a non-viral expression vector, e.g., a nucleic acid molecule that encodes for genes and/or regulatory elements necessary for the expression of genes. A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The terms “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Conservatively modified variants can include polymorphic variants, interspecies homologs (orthologs), intraspecies homologs (paralogs), and allelic variants.

The terms “identical” or percent “identity,” in the context of two or more nucleic acids or proteins, refer to two or more sequences or subsequences that are the same or have a specified percentage of nucleotides or amino acids that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST/. Such sequences are then said to be “substantially identical.” This definition also refers to, or may be applied to, the compliment of a test sequence. The definition also includes sequences that have deletions and/or additions, as well as those that have substitutions. Preferred algorithms can account for gaps and the like. Identity is typically calculated over a region that is at least about 25 amino acids or nucleotides in length, or more preferably over a region that is 50-100 amino acids or nucleotides in length, or over the entire length of a given sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

### III. Cells

The present inventors have shown that RF1 can be eliminated in bacteria without significantly affecting the growth rate or viability of the bacteria. The discovery can be exploited to reassign the UAG codon in bacteria, which is normally recognized by RF1 as a stop codon. The UAG codon can be reassigned to encode for any desired amino acid, naturally occurring or non-naturally occurring, for protein production.

RF1 can be eliminated in any bacterial species or strain, in particular, in those species and strains that retain a functional RF2 that efficiently recognizes the UAA and UGA stop codons. For example, as described herein, the K-12 strains of E. coli have an inefficient form of RF2 that has a 10-fold reduced recognition of UAA compared to RF2 in other bacteria (Uno et al. (1996) Biochimie 78:935). The K-12 strains are thus not ideal candidates for elimination of RF1 without genetic manipulation to correct for the RF2 inefficiency. Conversely, the B strains, which have a functional RF2, are quite amenable to reduction or elimination of RF1.

Thus, in some embodiments, the invention provides an RF1 deficient bacterial cell. Such bacteria are typically rendered RF1 deficient using recombinant methods, e.g., knockdown, as described herein, or using an inhibitory nucleic acid, e.g., antisense or siRNA. In some embodiments, the RF1 deficient bacteria retain less than 20% RF1 activity or expression, e.g., less than 10%, 8%, 5%, 2%, 1%, or less RF1 activity or expression, or RF1 activity or expression are undetectable. The RF1 deficient bacteria are, however, viable (able to grow). The lack of RF1 renders the bacteria deficient for recognizing the UAG codon as a stop codon. While the UAG stop codon recognition-deficient bacteria are deficient in recognizing the UAG codon as a stop codon relative to wild type bacteria, in some embodiments the bacteria are capable of recognizing the UAG codon as a sense codon (e.g., with misreading tRNAs or using orthogonal tRNA and synthetase).

In some embodiments, the bacterial cell includes a nucleic acid, e.g., a recombinant nucleic acid, which encodes RF2. In some embodiments, the cell includes a recombinant RF2 encoding nucleic acid which encodes an RF2 protein that includes a mutant (non-native) amino acid at position 246. The term “non-native” in the context of an amino acid at a specified position refers to an amino acid not found at the specified position in the wild-type cell.

RF2 activity in a particular bacterial species or strain can be determined as described in Uno et al., or Tuite and Stansfield (1994) Mol. Biol. Rep. 19:171. The level of RF2 activity can be compared to that of a Salmonella strain or a B strain E. coli. In some embodiments, the bacteria is selected for UAG reassignment (via RF1 deficiency) if the bacteria has an RF2 with at least 30, 40, 50, 60, 70, 80, 90, 95, 100, or higher percent activity compared to the activity of RF2 from a Salmonella strain or a B strain E. coli.

Examples of bacterial genera which can be used for the disclosed methods include Escherichia, Salmonella, Anaplasma, Butyrivibrio, Rhodococcus, Bifidobacterium, Laribacter, Pantoea, Mycobacterium, Glossina, Helicobacter, Synechococcus, Synechocystis, Caulobacter, Streptomyces, Rickettsia, Campylobacter, Neisseria, Arcobacter, Streptococcus, Staphylococcus, Yersinia, Bordetella, Candidatus, Chlamydia, Borrelia, etc. The sequences of RF1 and RF2 for these bacteria are publically available, e.g., from the NCBI website.

In some embodiments, the bacteria can include a variety of recombinant nucleic acids. In some embodiments, the bacterial cell can include a first exogenous recombinant nucleic acid which encodes a protein which includes a mutant amino acid (i.e., non-native, either naturally occurring or non-naturally occurring amino acid). In some embodiments, the first exogenous recombinant nucleic acid which encodes a protein which includes a plurality, e.g., 2, 3, 4, 5, 6, 7, 8, 9, 10 or more mutant amino acids. In some embodiments, the first exogenous recombinant nucleic acid is DNA, and the mutant amino acid is encoded by a TAG codon. In some embodiments, the first exogenous recombinant nucleic acid is RNA, and the mutant amino acid is encoded by a UAG codon. The term “mutant amino acid” refers an amino acid which is not present in a wild type sequence of the protein (e.g. a substitution or addition mutation). Exemplary mutant amino acids result from amino acid substitution at a specific position within the amino acid sequence of the protein. A specific amino acid substitution can be indicated by a variety of notations. For example, the term “XNNNY” refers to substitution of amino acid “X” at position “NNN” with amino acid “Y;” e.g., “A246T” refers to substitution with threonine (T) at position 246 in place of alanine (A), as does the term “Ala246Thr.” Further exemplary mutant amino acids result from the addition of one or more amino acids to the amino acid sequence of the protein.

In some embodiments, the bacterial cell can include a recombinant (e.g., exogenous) nucleic acid which encodes an orthogonal tRNA which includes a CUA anticodon. The CUA anticodon binds to a UAG codon, sot that a CUA anticodon will recognize a UAG codon.

In some embodiments, the bacterial cell can include a recombinant (e.g., exogenous) nucleic acid which encodes an orthogonal synthetase (also often referred to as a synthase) capable of functionally binding to an orthogonal tRNA as described herein. The term “functionally binding” refers to binding in the context of translational functionality. Accordingly, a functionally binding synthetase is capable of binding an orthogonal tRNA for the process of translation, e.g., associating the proper amino acid with the orthogonal tRNA.

In some embodiments, the recombinant nucleic acids described herein, or any combination thereof, can be included on a single expression vector for expression in bacteria. For example, a plasmid encoding the orthogonal tRNA and synthetase pair can be used as a single expression vector. The plasmid (or other expression vector) can also include additional recombinant nucleic acids, e.g., encoding for proteins that comprise a mutant amino acid, or encoding for a functional RF2.

In some embodiments, the bacterial cell can include exogenous recombinant nucleic acids, e.g., encoding proteins with 1, 2, 3, 4, 5, 6, 7, 8, 9, 10 or even more mutant amino acids. In some embodiments, the bacterial cell expresses a protein which includes 1 to 10 mutant amino acids, e.g., encoded by a UAG or TAG codon (RNA or DNA). In some embodiments, the cell includes a protein which includes 1 mutant amino acid encoded by a UAG or TAG codon.

### IV. Methods

The present disclosure provides methods for reassigning the meaning of the UAG stop codon in bacteria, e.g., by disrupting the expression or activity of release factor 1 (RF1). In some embodiments, the method comprises disrupting or removing the RF1 gene from the bacterial genome. In some embodiments, the method comprises reducing the expression of the gene encoding RF1, e.g., by introducing an inhibitory nucleic acid (antisense or siRNA) specific for RF1 to the bacteria. In some embodiments, the bacteria have a functional RF2. In some embodiments, the method further comprises introducing a recombinant nucleic acid encoding a functional RF2 protein (e.g., an RF2 protein from a Salmonella strain or a B strain E. coli).

Further provided are methods of producing a mutant protein, i.e., a protein comprising at least one mutant (non-native or non-wild type) amino acid, in a bacterial cell, as described above. The non-native amino acid can be a natural or non-naturally occurring amino acid. The method can include transfecting an RF1 deficient, viable bacterial cell with a recombinant nucleic acid encoding a protein comprising a mutant amino acid. The mutant amino acid can be encoded by a TAG codon where the first exogenous recombinant nucleic acid is DNA and by a UAG codon where the first exogenous recombinant nucleic acid is RNA. n some embodiments, the recombinant nucleic acid is DNA and the mutant amino acid is encoded by a TAG codon. In some embodiments, the recombinant nucleic acid is RNA and the mutant amino acid is encoded by a UAG codon.

In the case of non-naturally occurring amino acids, a number of possibilities are known in the art and available commercially (see, e.g., the Sigma-Aldrich catalog selection of Unnatural amino acid derivatives). Such amino acid mimetics and derivatives are often added to improve protein stability (e.g., pharmacological stability), to allow for easy detection, to add functionality (e.g., with an easily labeled or reactive side chain), to adjust protein structure, etc. In some embodiments, the non-naturally occurring amino acid corresponds to a synthetic or orthogonal tRNA/synthetase pair that can recognize and functionally interact with the non-naturally occurring amino acid. A brief list of non-naturally occurring amino acids includes: azetidine 2-carboxylic acid; D-phenylglycine; D-4-hydroxyphenylglycine; D-2-naphthylalanine; L-homophenylalanine; 2R,3S-phenylisoserine; D-cycloserine; 3,4-dehydrorproline; perthiaproline; canavanine; ethionine; norleucine; selenomethionine; aminohexanoic acid; telluromethionine; homoallylglycine; and homopropargylglycine. Additional examples can be found in the Sigma Aldrich catalog, as noted above, and in de Graaf et al. (2009) Bioconjugate Chem. 20:1281. Non-naturally occurring amino acids can also include those that are conjugated to a functional group, e.g., a detectable label or a PEG molecule.

The method can further comprise transfecting the cell with a recombinant nucleic acid encoding an orthogonal tRNA comprising a CUA anticodon. The method can further comprise transfecting the cell with a third exogenous recombinant nucleic acid encoding an orthogonal synthetase capable of functionally binding to the orthogonal tRNA. The bacterial cell is then allowed to express the protein under appropriate conditions (e.g., with appropriate media and temperature conditions, in the presence of the mutant amino acid), thereby producing the mutant protein.

### V. Kits

Further provided are kits for producing UAG reassigned bacteria, and for producing mutant proteins as described above.

An exemplary kit for producing a UAG reassigned bacterial cell can include:
A viable, RF1-deficient bacterial strain comprising a functional RF2;
An orthogonal tRNA/synthetase pair that recognizes UAG as a coding codon; and
An amino acid, representing the reassigned meaning of UAG, that is recognized by the orthogonal tRNA/synthetase.

An exemplary kit for producing a mutant protein in a bacterial cell can include:
A viable, RF1-deficient bacterial strain comprising a functional RF2;
A recombinant, exogenous nucleic acid encoding a protein comprising at least one mutant amino acid encoded by the UAG codon;
An orthogonal tRNA/synthetase pair that recognizes UAG as a coding codon; and
The mutant amino acid, representing the reassigned meaning of UAG, that is recognized by the orthogonal tRNA/synthetase.

Such kits can also include standard reagents for recombinant techniques, e.g., expression vector (e.g., for insertion of a mutant protein coding sequence), media, amino acids (e.g., including non-naturally occurring amino acids, or amino acid mimetics designed to interact with orthogonal tRNAs), nucleotide mixtures, buffers, etc. Kits often also include instructions for using components of the kits, e.g., for expressing mutant proteins. The kit can also include consumables, such as tubes, pipettes, and/or glassware for carrying out the methods of the invention.

The following discussion of the invention is for the purposes of illustration and description, and is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. All publications, patents, patent applications, Genbank numbers, and websites cited herein are hereby incorporated by reference in their entireties for all purposes.

### VI. Examples

### A. Methods

Strain Construction.

All strains in this study were created using the λ-red recombinase system (Datsenko, et al., Proc. Natl. Acad. Sci. U.S.A. 97, 6640-5 (2000); Tischer, et al., Biotechniques 40, 191-7 (2006)), and are described below.

JX2.0 and JX3.0 were constructed as follows. First, a mutagenesis cassette was generated using overlapping PCRs. This cassette contained a mutated form of prfB^{f}, a chloramphenicol resistance (Cm^{R}) cassette flanked by two I-Scel cut sites, and homologous regions on both the 5′ and 3′ end to facilitate recombination. The mutant prfB^{f} had the in-frame premature TGA element removed (Craigen et al., Proc. Natl. Acad. Sci. U.S.A. 82, 3616-20 (1985)), a Shine-Dalgarno like sequence mutated to a Sac II site, and the A246T mutation reverted back to alanine (Uno et al., Biochimie 78, 935-43 (1996)). This cassette was electroporated into MDS42 cells harboring the pKD46 plasmid (Datsenko and Wanner, Proc. Natl. Acad. Sci. U.S.A. 97, 6640-5 (2000)). Cm^{R} colonies were screened using PCR to verify a correct knock-in, and then by Sac II digestion to verify that the mutant prfB^{f} was present. The resultant strain was transformed with the plasmid pATBSR, a derivative of pACBSR that has a tetracycline resistance (Tet) cassette in place of the original Cm^{R} gene (Herring et al., Gene 311, 153-63 (2003)). Following induction with arabinose, cells were screened for removal of the Cm^{R} cassette using PCR and sequencing verification. Curing of the pATBSR plasmid resulted in the final JX2.0 strain.

JX3.0 was created using JX2.0 as the parental strain. JX2.0 cells harboring the pKD46 plasmid were electroporated with a PCR cassette to knock out the endogenous prfA gene. This cassette contained a Cm^{R} gene flanked by 5′ and 3′ homologous overhangs to facilitate recombination. Cm^{R} colonies were again screened by PCR and sequencing verification. The resultant strain, JX3.0, contains an exact replacement of prfA with the Cm^{R} gene. A Tet^{R} derivative of JX33 strain was constructed from JX33 by replacing the Cm^{R} cassette with a Tet^{R} cassette, and was used to express histone H3a.

JX2.0 and JX33 derivatives containing an N-terminal FLAG-tag in the yfiA and sufA genes were created as follows: A PCR cassette was synthesized using overlapping PCRs to yield a construct containing a 5′ homologous region followed by an I-SceI flanked kanamycin resistance (Kan^{R}) cassette and an N-terminal FLAG-tag appended onto the target gene, which itself serves as the 3′ overhang. In addition, immediately 5′ of the Kan^{R} cassette is 75 bp of DNA that is perfectly homologous to 75 bp on the 3′ of the Kan^{R} cassette. Use of this repeat element helps to leave a scarless insertion of the N-terminal FLAG tag upon excision of the Kan^{R} cassette. These constructs were electroporated into JX2.0 or JX33 cells containing the pKD46 plasmid. Kanamycin resistant clones were screened for insertions using PCR and sequence verified for FLAG-tag insertion. Resultant strains were transformed with pATBSR, induced with arabinose, and screened for removal of the Kan^{R} cassette. Sequence verified clones were then used for further studies.

To construct an MRA8 derivative harboring a prfB^{f}(A293E) gene identical to that in JX33, a construct was synthesized using overlapping PCRs harboring a full-length copy of prfB^{f}(A293E) from JX33 with a Kan^{R} cassette on the 3′ end. This construct was electroporated into MRA8 cells harboring the pKD46 plasmid, and kanamycin resistant clones were screened for insertion using PCR. Resultant strains were sequence verified and used for further analyses.

Knockout of the prfA gene was attempted using a chloramphenicol acetyltransferase (cat) cassette via established procedures (Datsenko & Wanner, Proc. Natl. Acad. Sci. U.S.A., 97, 6640-6645 (2000)). Briefly, 51 nucleotide overhangs homologous to the regions immediately 5′ and 3′ of prfA were appended to the cat gene. One microgram of this cassette was electroporated into various strains harboring the pKD46 plasmid, which expresses the phage λ red recombinase. Chloramphenicol resistant clones were screened for knockout by genomic PCR and any positive clones were verified by DNA sequencing and genomic sequencing.

DH10βf was constructed from DH10β as follows to revert the Thr246 in prfB back to Ala. A knockin cassette was first generated containing the prfB gene from BL21 (DE3) transcriptionally coupled to a kanamycin resistant (Kan^{R}) cassette. The Kan^{R} cassette was flanked on the 3′ end by a 51-nucleotide region homologous to the 3′ end of the endogenous prfB gene. One microgram of this cassette was electroporated into DH lop harbouring the pKD46 plasmid. Kan^{R} clones were screened using PCR and sequence verified for mutation of position 246 to Ala.

Growth Assay.

A colony was picked for each E. coli strain and grown overnight with appropriate antibiotic. Cells were normalized to an OD₆₀₀ of 1 and diluted 1:50 in fresh media and 1 mM unnatural amino acid (if applicable). For BL21 (DE3) strains, OD₆₀₀ was then measured every 30 min for 10 hr. For JX1.0 strains OD₆₀₀ was then measured every 60 min for 48 hr. Doubling times were then calculated from the exponential growth phase in each strain.

Plasmid Construction.

All plasmids were assembled by standard cloning methods and confirmed by DNA sequencing. pAIO plasmids containing EGFP gene with different TAG codons were synthesized as the following: EGFP cassettes with an N-terminal His6 tag, containing TAG's at various positions were created using overlapping PCRs. The following sites were used: Y182 for 1-TAG; Y39 and Y182 for 2-TAG; Y39, Y182 and Y151 for 3-TAG; Y39, Y74, Y143, Y151, Y182 and Y200 for 6-TAG; Y39, K101, D102, E132, D133, K140, E172, D173, D190 and V193 for 10-TAG; a 10 tandem TAG codons in place of E172 and D173 for 10-TAGtd. These cassettes were first cloned into pBP-Blunt (Biopioneer, San Diego, Calif.), and then digested and ligated into pBK-AIO vectors containing the orthogonal tRNA_{CUY}^{Tyr} and the M. jannaschii TyrRS (Wang, et al., Science 292, 498-500 (2001)) or the LW1RS (Wang, et al., Proc. Natl. Acad. Sci. U.S.A. 100, 56-61 (2003)) using Spe I and Bgl II.

Human histone H3a was expressed using two plasmids: pTak-tRNA-H3 and pBKt-ActKRS. The pTak plasmid contained the M. barkeri tRNA^{Pyl} and the human histone H3a gene with a His6 tag appended at the C-terminus. tRNA^{Pyl} was driven by the lpp promoter and terminated with the rrnC terminator. The gene for human histone H3a was codon-optimized using Gene Design (Richardson et al. (2006) Genome Res. 16:550), and synthesized by overlapping PCRs using multiple 40 bp primers. The optimized gene was cloned into pTak using Spe I and Bgl II sites, and was driven by the T5 promoter. Various mutant forms of histone H3a were then synthesized and also cloned into the pTak plasmid. The following histone H3a mutants were cloned: 1TAG—K9; 2TAG, K9 and K14; 3TAG—K9, K14, and K18; 4TAG—K9, K14, K18, and K23. The second plasmid pBKt-ActKRS expresses the ActK-specific synthetase. Six mutations (D76G, L266V, L270I, Y271F, L273A, and C313F) were introduced into the wild-type M. barkeri PylRS using overlapping PCR to generate the ActKRS. This cassette was digested with Nde I and Pst I and ligated into the precut pBK-JYRS vector (Wang & Schultz (2004) Angew Chem. Int. Ed. Engl. 44:34). The GlnRS promoter originally in pBK-JYRS was replaced with the trc promoter from pTrc (Invitrogen, Carlsbad, Calif.) to drive the expression of ActKRS.

GST was expressed with plasmids pVL-GST and pBK-LW1RS (Wang et al. (2003) Proc. Natl. Acad. Sci. 200:56). TAG codons were introduced at residue Y58 (1-TAG), Y58 and Y111 (2-TAG), Y58, Y111 and Y164 (3-TAG) of the Schistosoma japonicum GST gene. TAG-containing GST genes were cloned into pLEIZ using Spe I and Bgl II to afford pVL-GST. pVL-GST encodes the orthogonal tRNA_{CUA}^{Tyr} under the control of the lpp promoter and the rrnC terminator, and the GST(TAG) gene driven by the T5 promoter with a His6 tag appended at the C-terminus.

Western Analyses.

E. coli cells expressing EGFP plasmids were grown at 37° C. for 16 hours, harvested, washed 2× with PBS, and diluted to an OD600 of 0.1 in PBS. One mL cells were collected and resuspended in 100 μL L Blue Juice (Qbiogene, Carlsbad, Calif.) and incubated for 10 minutes at 95 C. Proteins were separated on 12% or 15% SDS polyacrylamide gel. After transfer, EGFP was detected using the HRP-conjugate penta-His antibody (Qiagen, Valencia, Calif.). Protein purification of YfiA and SufA was performed using established procedures with minor modifications. Purified YfiA and SufA were run on 15% or 20% SDS polyacrylamide gel, and detected by using a monoclonal FLAG M2 antibody (Sigma, St. Louis, Mo.). Blots were developed using the pico chemiluminescence kit (Thermo Scientific, Rockford, Ill.) according to manufacturer's specifications.

E. coli cells containing EGFP expression plasmids were grown at 37° C. for 16 hours, harvested, washed 2 times with PBS and diluted to an OD₆₀₀ of 0.1 in PBS. One milliliter of cells was collected and resuspended in 100 μL Blue Juice (Qbiogene, Carlsbad, Calif.) and incubated for 10 minutes at 95° C. Samples were separated by SDS-PAGE, transferred and probed with a penta-His antibody (Qiagen, Valencia, Calif.).

For Western analysis of YfiA, a modified version of an established protocol was used (Agafonov et al., EMBO Rep. 2, 399-402 (2001)). Briefly, one liter of E. coli cells harboring an N-terminal FLAG tagged yfiA gene were grown for 16 hours at 37° C. Cells were cold-shocked in ice-water for ten minutes followed by two hours of growth at 15° C. Cells were harvested by centrifugation and frozen at −80° C. SufA purification was also accomplished via an established procedure (Lee et al., Mol. Microbiol. 51, 1745-55 (2004)). For E. coli cells harboring an N-terminal FLAG tagged sufA gene, a 50 mL culture was grown for 16 hours at 37° C. Cells were diluted to an OD₆₀₀ of 0.02 in one liter of fresh media. Once the OD₆₀₀ reached 0.2, phenazine metholsulfate (Sigma, St. Louis, Mo.) was added to a final concentration of 0.1 mM. Cells were harvested by centrifugation and pellets were frozen at −80° C. after 90 minutes of growth at 37° C. Protein from both cell types was extracted using BPER reagent (Thermo Scientific, Rockford, Ill.) and then applied to an Anti-FLAG agarose column (Sigma) to remove the vast majority of contaminating protein. Purified protein was then visualized using Western blotting with the monoclonal FLAG M2 antibody (Sigma).

In-Cell Fluorescence Assay.

In-cell fluorescence intensity was determined using a FluoroLog-3 (Horiba Jobin Yvon). E. coli colonies were picked and grown 16 hours with or without unnatural amino acids. Cells were washed two times in PBS buffer, and diluted in PBS to an OD₆₀₀ of 0.1. The emission spectrum of EGFP was measured using an excitation wavelength of 488 nm scanning from 503 to 560 nm. Fluorescence intensity of each sample was compared using the intensity at the maximal emission at 511 nm. Slit widths and integration times remained constant between all readings.

Protein Purification.

For EGFP preparations, 100 or 500 mL cultures were grown for 16 hr with or without unnatural amino acid. Cells were pelleted, resuspended in lysis buffer (10% glycerol, 50 mM Tris pH 8.0, 500 mM NaCl, 5 mg/mL lysozyme, DNase, and 10 mM β-mercaptoethanol) and sonicated for 4 cycles at 90% power with a duty cycle of 50. Cell lysate was collected after centrifugation at 12,000 g for 30 min. Lysate was then added to 500 μL of pre-equilibrated Ni-NTA resin (Qiagen, Valencia, Calif.), washed with 50 column volumes of wash buffer (50 mM Tris, 500 mM NaCl, 20 mM imidazole, pH 8) and then eluted in three 1 mL fractions of elution buffer (same as wash buffer except 250 mM imidazole). Purified EGFP was buffer exchanged to 50 mM Tris buffer containing 500 mM NaCl (pH 8.0) using Microcon Ultracel YM-10 spin columns (Millipore, Billerica, Mass.), and further purified using a Sephadex-200 size exclusion column on a UPC-900 FPLC (GE healthcare, Piscataway, N.J.). Peak fractions were analyzed by SDS-PAGE and pooled for further analysis.

For histone H3a preparations, E. coli colonies transformed with plasmids pTak-tRNA-H3 and pBKt-ActKRS were picked and grown 16 hours. Cells were diluted 1:100 into fresh media containing 5 mM ActK or 1 mM pActF. For all ActK preparations, nicotinamide was added to a final concentration of 5 mM to minimize deacetylation. When the OD₆₀₀ reached 0.5, cells were induced with 0.4 mM of IPTG and grown for 4 hours at 37° C. Cells were pelleted and frozen overnight to facilitate lysis. Pellets were thawed for ten minutes in a water bath, and then processed as described (Luger et al. (1999) Methods Enzymol. 304:3). The final cell lysate was collected and applied to a Ni-NTA column pre-equilibrated with wash buffer (6 M guanidine HCl in PBS pH 7.6, 25 mM imidazole). Lysate was applied to the column 2 times, followed by 20 times the column volume of wash buffer. Fractions containing Histone H3a were eluted with elution buffer (wash buffer plus 250 mM imidazole) and analyzed by SDS-PAGE and Western blot.

All protein concentrations and total yields were determined using the Bio-Rad protein assay kit (Hercules, Calif.) according to manufacturer's specifications.

Mass Spectrometry.

Intact proteins were analyzed by ESI-MS using a LTQ Velos mass spectrometer (Thermo Scientific, Rockford, Ill.). Automated 2D nanoflow LC-MS/MS analysis was performed using LTQ tandem mass spectrometer (Thermo Electron Corporation, San Jose, Calif.).

Intact protein analysis by ESI-MS: Purified pActF-containing EGFP proteins were dissolved in 1% formic acid and infused into a LTQ Velos mass spectrometer at 1 μL/min by a syringe pump. MS scans were collected for 1 minute. About 1,600 MS spectra were collected for each sample. Spectra were averaged and the charge states were de-convoluted using a freeware MagTran (Zhang and Marshall, J. Am. Soc. Mass Spectrom. 9, 225-33 (1998)).

Tandem MS analysis: Purified pActF-containing EGFP, SufA and YfiA proteins were solubilized in 50 mM HEPES (pH 7.2). The proteins were reduced and alkylated using 1 mM Tris(2-carboxyethyl)phosphine (Fisher, AC36383) at 95° C. for 5 minutes and 2.5 mM iodoacetamide (Fisher, AC12227) at 37° C. in dark for 30 minutes, respectively. pActF-containing EGFP was digested with 1:50 chymotrypsin (Roche, 11418467001). SufA was digested with 1:50 trypsin (Roche, 03708969001) and YfiA was digested by both trypsin and Lys-C (Roche, 11420429001) at 37° C. overnight. Automated 2D nanoflow LC-MS/MS analysis was performed using LTQ tandem mass spectrometer (Thermo Electron Corporation, San Jose, Calif.) employing automated data-dependent acquisition. The detailed LC-MS/MS method can be found in Tanner, et al., Genome Res. 17, 231-9 (2007); Castellana, et al., Proc. Natl. Acad. Sci. U.S.A. 105, 21034-8 (2008). Briefly, the peptides were fractionated by the on-line SCX column using a series of 7 salt gradients (10 mM, 20 mM, 30 mM, 50 mM, 70 mM, 100 mM, and 1M ammonium acetate for 20 minutes), followed by high resolution reverse phase separation using an acetonitrile gradient of 0 to 80% for 120 minutes.

The full MS scan range of 400-2000 m/z was divided into 3 smaller scan ranges (400-800, 800-1050, 1050-2000) to improve the dynamic range. Both CID (Collision Induced Dissociation) and PQD (Pulsed-Q Dissociation) scans of the same parent ion were collected for protein identification and quantitation. Each MS scan was followed by 4 pairs of CID-PQD MS/MS scans of the most intense ions from the parent MS scan. A dynamic exclusion of 1 minute was used.

The raw data was extracted and searched using Spectrum Mill (Agilent, version A.03.02). The CID and PQD scans from the same parent ion were merged together. MS/MS spectra with a sequence tag length of 1 or less were considered as poor spectra and discarded. The rest of the MS/MS spectra were searched against the NCBI (National Center for Biotechnology Information) RefSeq protein database (version 21, January 2007) limited to E. coli (16,324 sequences) plus the SufA and YfiA protein sequences with extended C-terminal sequences, as well as EGFP protein sequence. The enzyme parameter was limited to full chymotrypsin, tryptic or Lys-C peptides with a maximum miscleavage of 1. All other search parameters were set to the SpectrumMill default settings (carbamidomethylation of cysteines, +/−2.5 Da for precursor ions, +/−0.7 Da for fragment ions, and a minimum matched peak intensity of 50%). A variable modification of Gln to pActF (+61 Da) was used for pActF-containing EGFP database search. MS/MS spectra were validated using a cutoff score as follows: 1+peptide>9; 2+peptide>9; 3+peptide>12.

Trypsin-digested protein was analyzed by LC electrospray ionization MS as described (Schubert, D. et al., J. Neurochem., 109, 427-435 (2009)). Briefly, samples were loaded onto a capillary column with integrated spray tip (75 μm I.D., 10 μm tip, New Objective, Woburn, Mass.), which was packed in-house with C18 reversed phase material (Zorbax SB-C18, 5 μm particle size, Agilent, Santa Clara, Calif.) to a length of 10 cm. The reversed phase elution was achieved by a linear gradient of 0-60% acetonitrile in 0.1% formic acid within 60 min at a flow rate of 300 nL/min. The eluate was introduced into a Thermo LTQ-Orbitrap mass spectrometer (ThermoFisher, Waltham, Mass.) via a nano-spray source. Mass spectrometric analysis was conducted by recording precursor ion scans at a resolution of 60,000 in the Orbitrap Fourier-transform analyzer followed by MS/MS scans of the top 5 ions in the linear ion trap (cycle time approx. 1 s). An active exclusion window of 90 s was employed. Data were analyzed on a Sorcerer Solo system running Sorcerer-Sequest and by using the Mascot algorithm (V. 27 rev. 11, Matrix Science, London, UK). Data were further analyzed and visualized using the Scaffold software package (v. 2.6, Proteome Software).

Genomic Sequencing of E. coli Strains.

Genomic DNA from JX2.0, JX3.0, and JX33 was harvested and purified using a Qiagen DNeasy kit. One μg of genomic DNA was used to prepare DNA libraries for sequencing. Genomic DNA was fractionated using the Covaris S2 System (Applied Biosystems, Foster City, Calif.) using the following parameters: cycle number=6, duty cycle=20%, Intensity=5, cycles/burst=200 and time=60 seconds. Fractionated DNA was purified using a Qiagen PCR minielute purification kit. Purified DNA was repaired using the Epicentre End-It Repair Kit (Madison, Wis.) and purified using a Qiagen minielute column. Purified DNA was A-tailed using dATP and Klenow (3′-5′ exo-) from New England Biolabs (NEB, Ipswitch, Mass.) and then purified with a Qiagen minielute column. Libraries were prepared using the NEBNext DNA Sample Prep Reagent Set 1 (New England Biolabs), following recommended protocols. Purified DNA was then ligated overnight with Illumina genomic DNA adapters using T4 DNA Ligase from NEB and purified using a Qiagen minielute column. The ligated DNA was run on a 2% agarose gel and size selected to remove adapters. Gel extraction was performed on the gel slice using Qiagen minielute gel purification kit. Purified DNA was PCR amplified using 1 μL of ligated DNA and Phusion Taq from NEB and size selected from a 2% agarose gel.

Genomic DNA libraries were sequenced using the Illumina Genome Analyzer II (Illumina, San Diego, Calif.) as per manufacturer's instructions. Sequencing of genomic DNA libraries was performed up to 82 cycles. Image analysis and base calling were performed with the standard Illumina pipeline (Firecrest v1.3.4 and Bustard v.1.3.4).

Sequence alignments and SNP analysis were performed using the SHORE package (Ossowski, et al., Genome Res. 18, 2024-33 (2008)) according to the documentation provided with the software. In brief, the E. coli K-12 MG1655 reference genome was preprocessed into a SHORE acceptable format. Next, FASTQ files for each sample were converted to a SHORE flat file format. Reads were mapped using Genomemapper contained within the SHORE package using the following parameters −n 4, −g 3. Capitalizing on the large amount of coverage for this experiment we identified large deletions. We posited that any region of the reference genome where reads did not map were regions that were deleted in this strain. Therefore, we subtracted all positions from the reference genome that were covered by at least one read. The set of positions left over were the ones we called deleted. From this analysis the only deletion different between the two strains was the prfA gene. FASTQ files for each sample have been deposited to the Short Read Archives (SRA Accession# SRA016379.1).

Statistics.

Statistical analysis of the GCT-GAA knock-in data was performed using Fisher's exact test on Prism software (GraphPad Software, La Jolla, Calif.).

### B. Example 1

### Generation of RF1 Deficient E. coli Strains

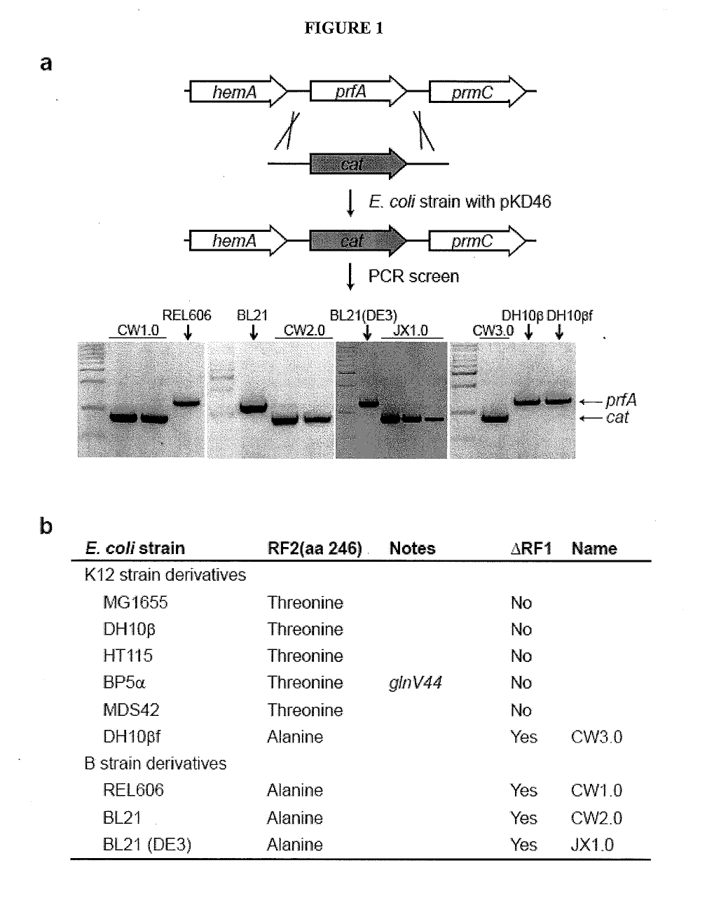

To determine if RF1 is essential in E. coli, we tried to replace the RF1-encoding gene, prfA, with the chloramphenicol acetyltransferase gene in a variety of strains (FIG. 1) using a λ red recombinase-based homologous recombination method (Datsenko & Wanner, Proc. Natl. Acad. Sci. U.S.A., 97, 6640-6645 (2000)). E. coli K-12 and B are classes from which most E. coli strains are derived (Studier et al., J. Mol. Biol., 394, 653-680 (2009)). We initially attempted to knock out RF1 in three common K-12 strains MG1655, DH10β and HT115. Knockout of RF1 was assayed by PCR to amplify the gene at the prfA locus (FIG. 1a). The RF1 knock out was unsuccessful in all three K-12 strains (FIG. 1b). We next attempted the RF1 knockout with two additional K-12 strains with features related to translational termination. The BP5α strain harbors a glutaminyl amber suppressor tRNA, which makes the UAG codon ambiguous between a stop signal and Gln. The BP5α strain was included to determine whether stop codon ambiguity is a factor to prime RF1 removal. The MDS42 strain has nearly 700 nonessential genes deleted (Posfai et al., Science, 312, 1044-1046 (2006)), and was included to determine whether reduced genome size and thus termination load can allow for RF1 removal. RF1 knockout was, however, also unsuccessful in these K-12 strains, indicating that an amber suppressor tRNA and a minimal genome did not allow for RF1 removal in K-12.

K-12 strains have a Ala246Thr mutation in its RF2 gene prfB, reducing RF2 recognition of UAA 10-fold (Uno et al., Biochimie, 78, 935-943 (1996)). The UAA codon accounts for the termination of ˜64% of E. coli genes (Nakamura et al., Nucleic Acids Res. 28, 292 (2000)). The Ala246Thr mutation likely impairs the ability of RF2 to efficiently recognize UAA stop codons, so that the mutation prevents viability of RF1 knockout cells.

E. coli B strains have the wild type RF2 with an alanine at position 246 (or at a position corresponding to amino acid 246 of SEQ ID NO:2) (Studier et al., J. Mol. Biol., 394, 653-680 (2009)). We thus generated RF1 knockouts in three common B strains, which successfully resulted in viable cells. RF1 knockouts were generated from B strains REL606, BL21 and BL21(DE3), generating strain CW1.0, CW2.0 and JX1.0, respectively (FIG. 1a). In addition, to determine if the Ala246Thr mutation in RF2 prevents viability of RF1 knockouts in K-12 strains, we reverted the 246Thr mutation to Ala in the K-12 strain DH10β (generating the DH10βf strain). DH 10βf also permitted the direct knockout of RF1 (FIG. 1a), demonstrating that RF1 is not essential in E. coli, in contrast to the previous conclusions, as long as a functional RF is present in the bacteria.

Full genomic sequencing was performed on RF1 knockout strains JX1.0, CW 1.0, CW2.0 and CW3.0, and compared to the respective parental BL21(DE3), REL606, BL21, and DH10β strains (Jeong et al., Mol. Biol., 394, 644-652 (2009); Durfee et al., J. Bacteriol., 190, 2597-2606 (2008)). RF1 deletion was verified in all cases. For CW2.0, no other mutations were found throughout the genome. For JX1.0, only a few additional single nucleotide polymorphisms (SNPs) were found (Table 1). Most of the SNPs are silent mutations in genes that are of phage origin. None of the SNPs correspond to known mutations that would complement an RF1 deficiency (Zhang et al., J. Mol. Biol., 242, 614-618 (1994); Ito et al., Proc. Natl. Acad. Sci. U.S.A., 95, 8165-8169 (1998); Dahlgren & Ryden-Aulin, Biochimie, 82, 683-691 (2000); Kaczanowska & Ryden-Aulin, J. Bacteriol. 186, 3046-3055 (2004)). The results indicate that RF1 was knocked out from the parental E. coli strains without incurring additional, potentially compensatory, mutations.

TABLE 1
Single nucleotide polymorphisms between
the JX1.0 and parental BL21 (DE3) strains

| | Refer- | | | | | |
|---|---|---|---|---|---|---|
| Position | ence¹ | Read | Number | Conf | Gene | Mutation |
| 1200795 | T | C | 382 | 1 | (2) | |
| 1200805 | C | T | 385 | 1 | (2) | |
| 1200849 | C | T | 409 | 1 | (2) | |
| 1200862 | C | T | 397 | 1 | ycgX | Silent |
| 1200961 | A | C | 196 | 1 | ycgX | Silent |
| 1200982 | T | C | 217 | 1 | ycgX | Silent |
| 2530079 | G | C | 17 | 0.809 | (3) | |
| 3708564 | T | C | 422 | 1 | ECD_03520⁴ | Silent |

¹Reference genome for BL21 (DE3) is As# CP001509 (Jeong 2009).
(2) These Mutations lie in the intergenic region between ycgX and ycgW.
(3) This mutation lies in the intergenic region between suhB and yfhR.
⁴ECO_03520 is a conserved gene involved in transport of hexuronate.

### C. Example 2

### UAG in RF1 Deficient E. coli JX1.0 Strain

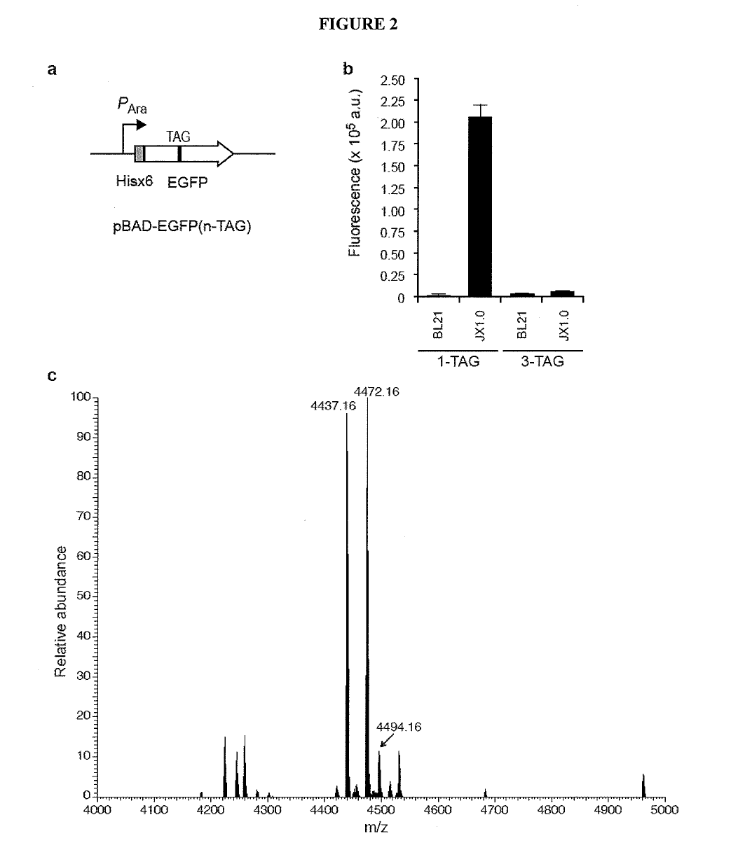

To determine how E. coli cells would interpret the UAG codon in the absence of RF1, we mutated 1 and 3 tyrosine codons to UAG in the EGFP gene to make 1-TAG and 3-TAG EGFP reporter, respectively. The expression of 1-TAG and 3-TAG were tested using a pBAD plasmid in BL21(DE3) and JX1.0 (FIG. 2a). BL21(DE3) cells expressing either 1- or 3-TAG reporter showed no GFP fluorescence, indicating that RF1 acted normally to terminate EGFP translation at UAG. JX1.0, on the other hand, demonstrated a high level of fluorescence from the 1-TAG mutant, and a lower, but reproducible level of fluorescence from the 3-TAG mutant (FIG. 2b). The results indicate that endogenous tRNAs can suppress the UAG codon in JX1.0. To reveal these tRNAs, EGFP protein with the 1-TAG reporter expressed in JX1.0 was purified, and the amino acid incorporated at the UAG site was identified using mass spectrometry (FIG. 2c). Tyr, Gln, and Trp were found incorporated at the UAG site. To ensure that this is not from a suppressor mutation in the endogenous tRNAs, chromosomal tRNA^{Tyr}, tRNA^{Gln}, and tRNA^{Trp} were resequenced in JX1.0, and all were found to be wild type. The anticodons of these three tRNAs have only a single base mispairing with the UAG, and these tRNAs are known to weakly interact with the UAG codon (Murgola, in tRNA: Structure, Biosynthesis, and Function (eds. Soll & RajBhandary) 491-509 (ASM Press, Washington, D.C., 2005)). The level of UAG misreading is not apparent in the presence of RF1 in BL21(DE3), but can be observed in JX1.0 in the absence of RF1.

### D. Example 3

### Reassignment of the UAG Codon Using Orthogonal tRNA/Synthase Pair in JX1.0

Unconditional knockout of RF1 allows for reassignment of the UAG codon. We sought to reassign the meaning of UAG to code for an amino acid in JX1.0, similar to the evolutionary pathway proposed for ciliates. An orthogonal tRNA/synthetase pair, the tRNA tRNA_{CUA}^{Tyr}/LW1RS pair, was introduced into JX1.0. This pair does not crosstalk with endogenous E. coli tRNA/synthetase pairs (Wang et al., Science, 292, 498-500 (2001)). The tRNA_{CUA}^{Tyr} decodes the UAG codon specifically through its anticodon CUA. LW1RS is engineered to charge the tRNA_{CUA}^{Tyr} with an unnatural amino acid p-acetylphenylalanine (pActF) (Wang et al., Proc. Natl. Acad. Sci. U.S.A., 100, 56-61 (2003)).

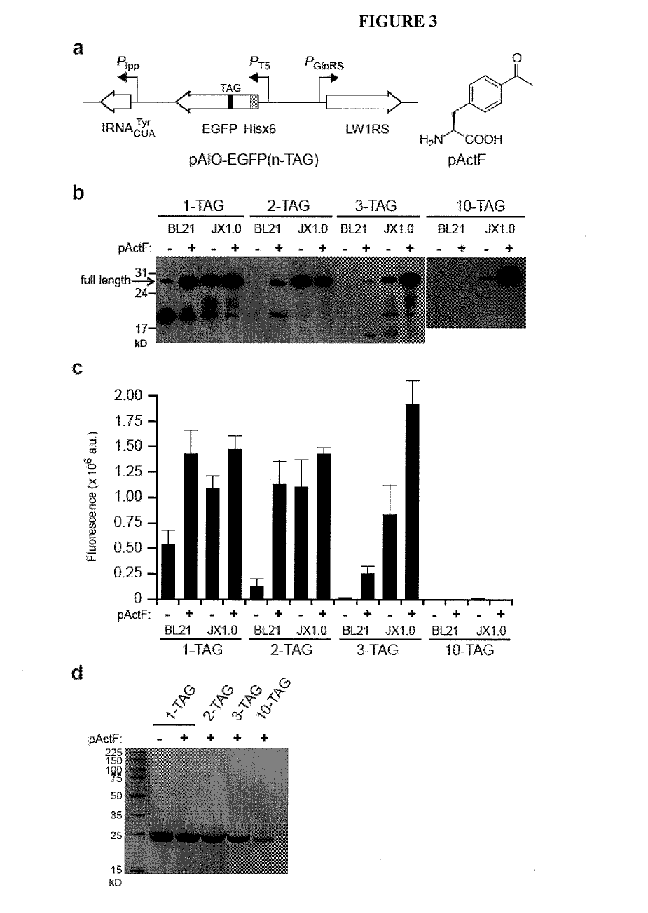

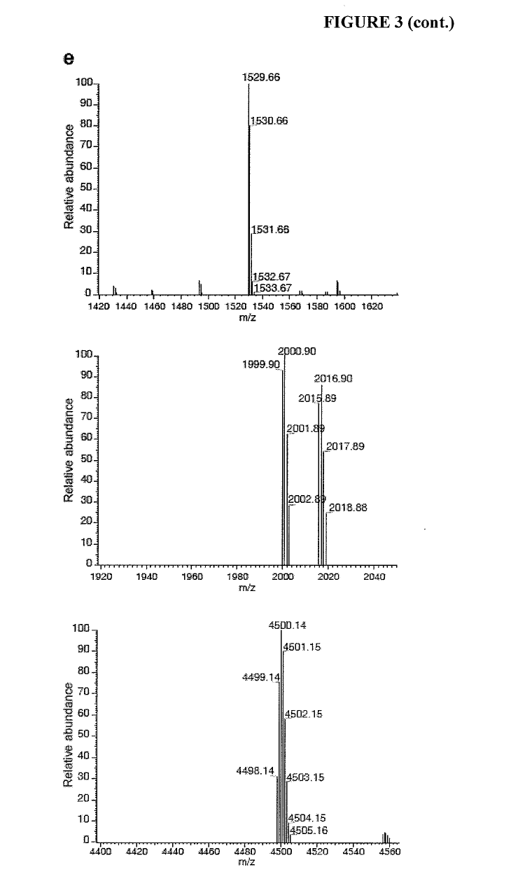

An EGFP gene containing 1-, 2-, 3-, or 10-UAG codons was co-expressed with the tRNA_{CUA}^{Tyr}/LW1RS in a single plasmid pAIO-EGFP(n-TAG) (FIG. 3a). EGFP expression was assayed by Western blotting (FIG. 3b) and in-cell fluorescence (FIG. 3c). When pActF was not added to the growth media, BL21(DE3) cells expressed small amount of full-length EGFP with the 1-TAG reporter only, suggesting that the tRNA_{CUA}^{Tyr}/LW1RS pair incorporates a natural amino acid in low efficiency in the absence of pActF. No full-length EGFP was detected for 2-, 3-, and 10-TAG reporter. When pActF was supplied in the growth media, BL21(DE3) cells showed efficient expression of the 1-TAG EGFP mutant, but EGFP expression decreased precipitously with the addition of each UAG due to the competition from RF1. The use of 3-UAG codons in EGFP virtually abolished protein expression, and no protein could be detected at all in the 10-UAG mutant.

In contrast, JX1.0 expressed full-length EGFP for 1-, 2-, 3-, and 10-TAG mutants, although the efficiency decreased with the number of UAG codons. We then purified the EGFP protein from JX1.0 expressing the pAIO-EGFP (1-TAG) in the absence of pActF (FIG. 3d), and analyzed it with mass spectrometry. Consistently, Tyr, Gln, and Trp were found at the UAG site as observed in FIG. 2c, confirming the misreading of UAG by endogenous tRNAs in JX1.0. In the presence of pActF, JX1.0 showed high expression of EGFP in all mutants as indicated by Western (FIG. 3b) and in-cell fluorescence (FIG. 3c). EGFP proteins were purified in the yields of 8.5, 7.1, 9.7, and 1.2 mg/L for the 1-, 2-, 3-, and 10-TAG EGFP samples respectively (FIG. 3d). There was no decrease in incorporation efficiency when the UAG codon was increased from 1, 2, to 3, indicating that UAG was efficiently reassigned to a sense codon in JX1.0. The reduced yield in the 10-TAG EGFP sample is likely because 10 pActF interfere with EGFP folding and/or stability. The reduced fluorescence of the 10 pActF-EGFP (FIG. 3c) supports this.

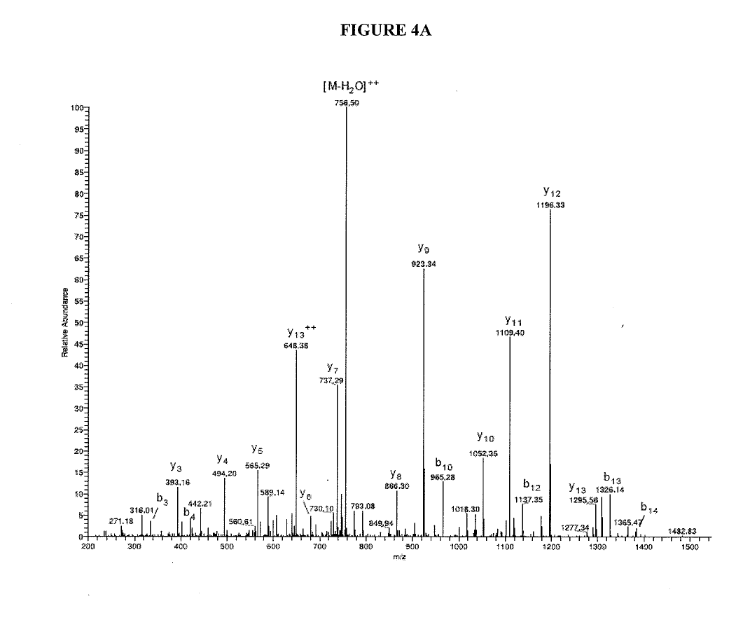

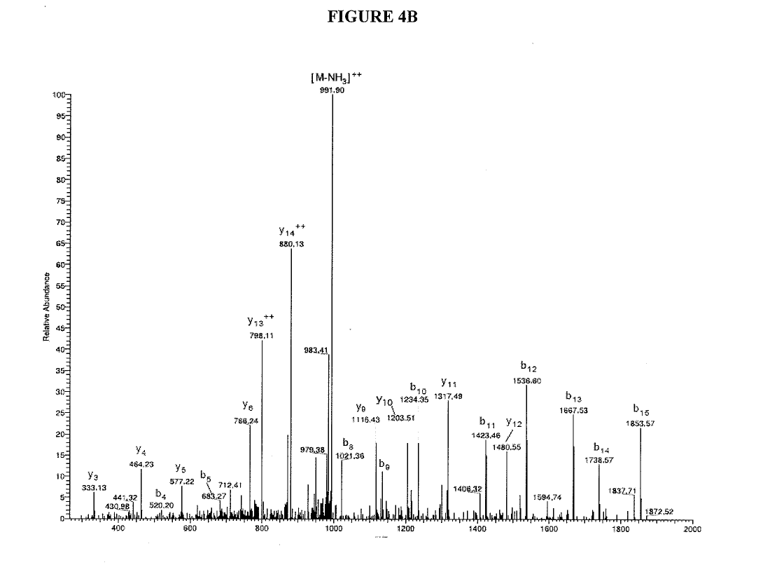

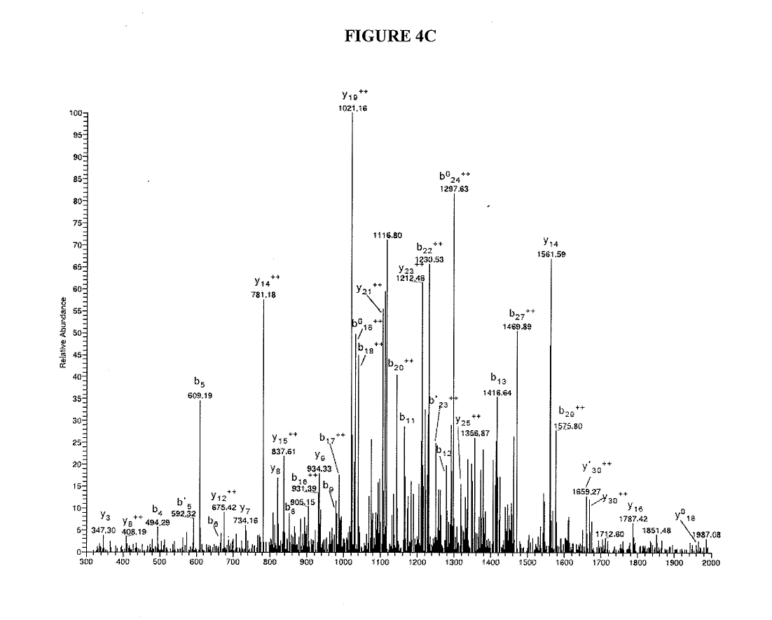

To confirm that pActF was incorporated at the UAG site, we purified the EGFP protein expressed in JX1.0 using pAIO-EGFP (3-TAG) in the presence of pActF. The EGFP was analyzed with mass spectrometry (FIG. 3e). The monoisotopic masses of the tryptic peptides demonstrate that pActF was incorporated at all three UAG sites. No peaks corresponding to other amino acids at the UAG sites were detected. The precursor ions of the peptides containing the UAG sites were individually fragmented with an ion trap mass spectrometer. The fragment ion masses were unambiguously assigned, confirming that pActF was incorporated the UAG sites (FIG. 4). These results indicate that missuppression of UAG in JX1.0 by endogenous tRNAs were outcompeted by the tRNA_{CUA}^{Tyr}/LW1RS pair, which specifically decodes UAG as pActF.

### E. Example 4

### JX1.0 Cells are Viable

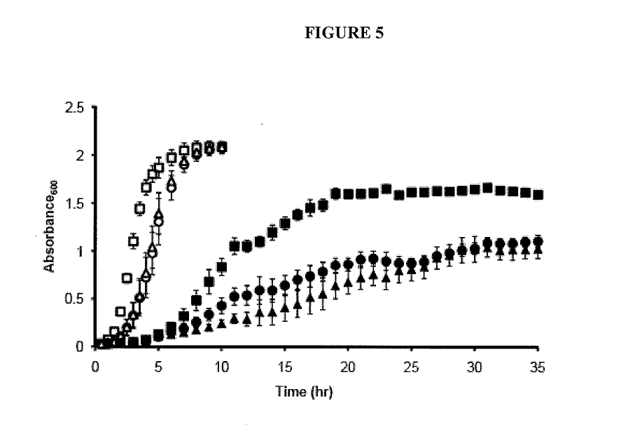

To evaluate the response of E. coli to RF1 deletion and subsequent UAG reassignment, we assessed the health of JX1.0 using a growth assay. JX1.0 was healthy, cloneable, and stable in culture; no changes in phenotype or genotype were observed after growing over 20 generations. Compared to the parental BL21 (DE3), JX1.0 showed a slower growth rate (FIG. 5). The doubling time for JX1.0 was 91 minutes compared to 25.5 minutes for the BL21(DE3). This suggests that RF1 deficiency, lack of proper termination, and reduced misreading of UAGs by near-cognate tRNAs burdens the cell, but these factors are not lethal as previously reported. Introduction of the pAIO plasmid expressing the orthogonal tRNA_{CUA}^{Tyr}/LW1RS pair in the absence of pActF increased the doubling of JX1.0 to 135 minutes and of BL21(DE3) to 41 minutes, potentially due to toxicity of unacylated tRNAs. The addition of pActF to the growth media together with the expression of tRNA_{CUA}^{Tyr}/LW1RS further increased the doubling time to 253 minutes for JX1.0, while not affecting BL21(DE3). The reduced growth rate of JX1.0 can likely be explained by efficient incorporation of pActF at UAG positions, which adds to energy expended by protein translation. RF1 in BL21(DE3) simply terminates translation, and thus reduces protein translation pressure.

### F. Example 5

### Generation of RF1 Deficient Strain JX3.0

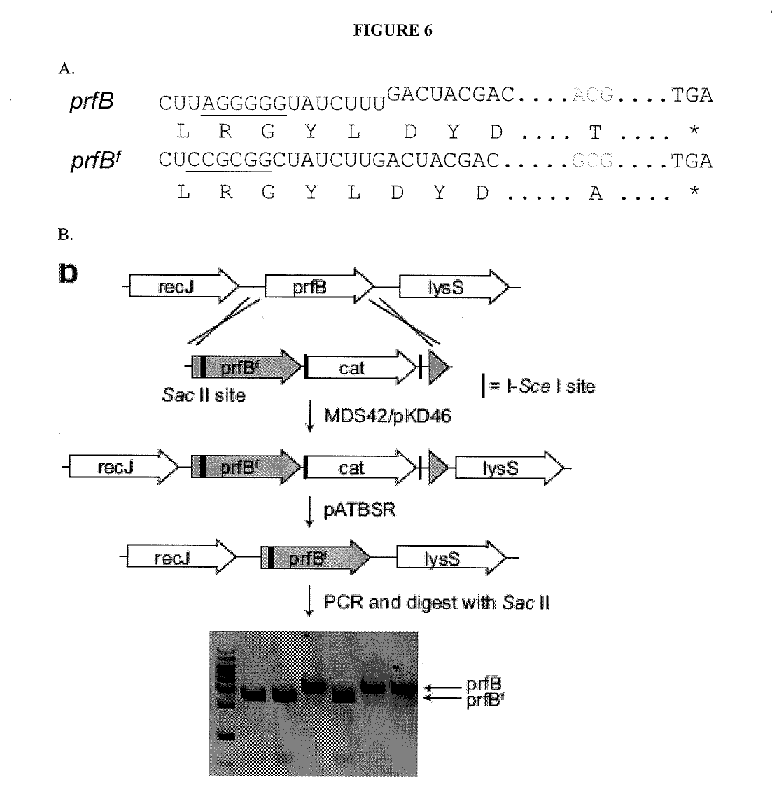

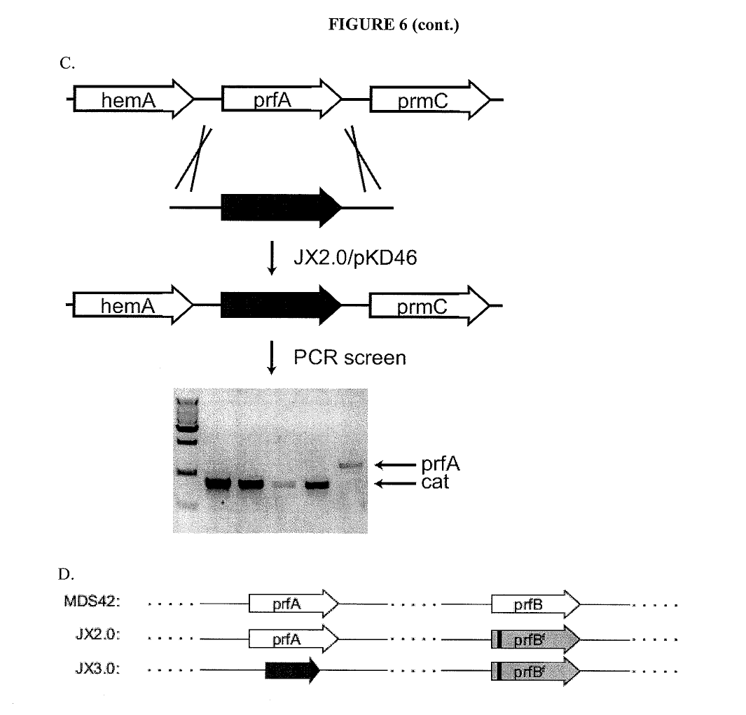

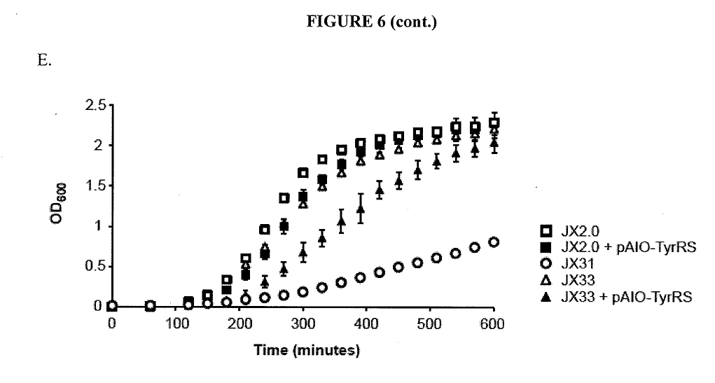

RF2 expression in E. coli is tightly autoregulated by an in-frame UGA codon in its mRNA that requires a +1 frameshift to generate full-length RF2 (Craigen et al., Proc. Natl. Acad. Sci. U.S.A. 82, 3616-20 (1985)). As explained above, K-12 E. coli strains include a Ala246Thr mutation in RF2 that reduces its recognition of UAA. We used the E. coli strain MDS42 as the parental strain due to its reduced genome (Posfai et al., Science 312, 1044-6 (2006)), and removed the in-frame UAG autoregulation element and reverted residue 246 back to Ala in the prfB^{f} gene (FIG. 6A). The gene encoding RF2, prfB, was first replaced by the prfB^{f} gene coupled to a Cm^{R} cassette in MDS42 cells using the λ-red recombination system (FIG. 6B). The Cm^{R} cassette was subsequently excised from the Cm resistant clones using the pACBSR plasmid system developed for markerless insertions (Tischer, et al., Biotechniques 40, 191-7 (2006)). The resultant strain, JX2.0, has the prfB replaced by prfB^{f}. Knockout of prfA was then attempted in JX2.0 by electroporating the linear Cm^{R} knock-in cassette with flanking sequence identical to those of prfA (FIG. 6C). Genomic PCR screening of Cm resistant colonies showed that they contained the Cm^{R} cassette at the endogenous prfA locus, indicating that RF1 was indeed knocked out (FIG. 6C). RF1 knockout was further confirmed with genomic sequencing, and the strain was called JX3.0 (FIG. 6D).

The growth rates of JX2.0 and JX3.0 cells were compared (FIG. 1E). JX2.0 grew with a doubling time of 27.1 minutes in the Luria-Bertani media, similar to the parental DH10β. JX3.0 doubled every 74.4 minutes, indicating that the RF1 knockout slows cell growth. The JX3.0 cells are, however, viable and sustainable, demonstrating that RF1 is not essential for survival. A single JX3.0 colony, JX33, grew faster than others with a doubling rate (27.5 minutes), similar to that of JX2.0.

### G. Example 6

### UAG in RF1 Deficient E. coli JX3.0 Strain

Deletion of RF1 from E. coli presumably reassigns the UAG codon to a blank codon. As with JX1.0, introduction of an orthogonal tRNA/synthetase pair which recognizes the UAG codon in JX3.0 would translate UAG with the amino acid for which the synthetase is specific, essentially reassigning UAG to a sense codon. the fast-growing form of JX3.0, the JX33 strain, was used to show that, in the absence of RF1 competition, incorporation efficiency at UAG would increase.

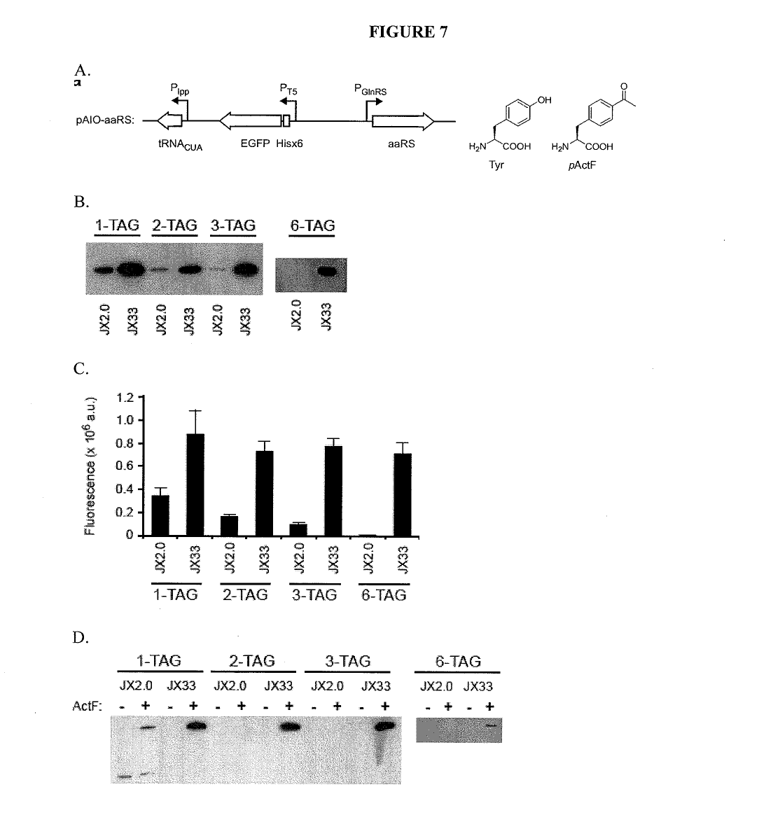

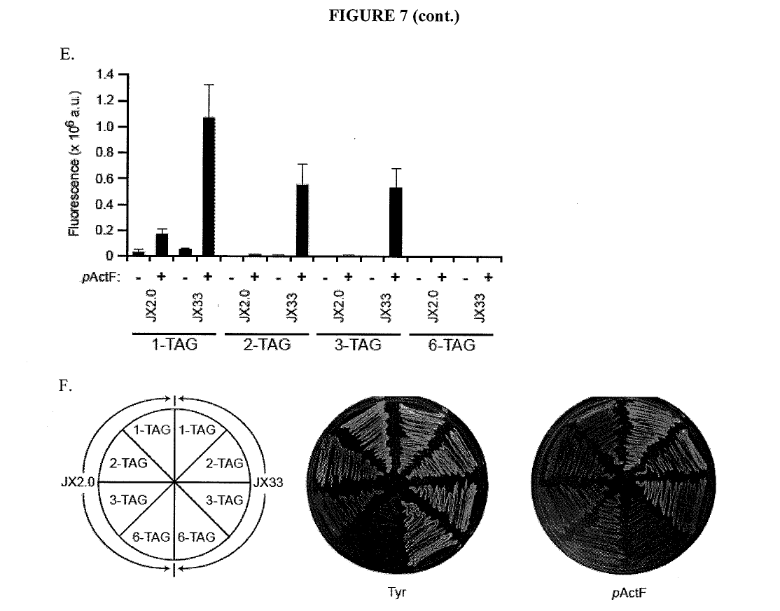

Similar to Example 3, we used a single All-in-One expression plasmid (pAIO) with an orthogonal amber suppressor tRNA, an orthogonal aminoacyl-tRNA synthetase, and an EGFP reporter with an N-terminal hexahistidine (His6) (SEQ ID NO:5) tag (FIG. 7A). TAG mutations were introduced into tyrosyl sites in the EGFP gene to create 1-, 2-, 3-, and 6-TAG EGFP reporters. We first tested incorporation efficiency at UAG sites using the orthogonal tRNA_{CUA}^{Tyr}/TyrRS derived from archaebacterium Methanococcus jannaschii (Wang et al., Science 292, 498-500 (2001)), which inserts a tyrosine residue at UAG codons. In parental JX2.0 cells, EGFP protein yields were reduced with each additional UAG, and no full-length EGFP in the 6-TAG reporter was detected by Western blot (FIG. 7B).

In contrast, JX33 showed increased levels of protein and no reduction in EGFP protein yields across all TAG mutants.

For EGFP with a single TAG site, JX33 protein expression was 254% of that in JX2.0. For EGFP with 6 TAG sites, JX2.0 yielded no protein whereas JX33 afforded 6.8 mg/L, which is only about a two-fold reduction in comparison to wild-type EGFP expressed from the same plasmid system without any TAG mutations. In-cell fluorescence intensity was measured for each mutant using fluorometry for both the JX2.0 and JX3.0 strains. In JX2.0, fluorescence intensity decreased with each additional TAG, while JX3.0 fluorescence was similar among all mutants and much higher than in JX2.0 (FIGS. 7C and 7F). These results demonstrate that the JX33 strain has much higher incorporation efficiency for tyrosine at UAG sites than the parental JX2.0 strain. The knockout of RF1 also allows multiple UAG sites to be efficiently suppressed with tyrosine in JX33, unlike in the RF1-containing JX2.0 strain.

### H. Example 7

### Reassignment of UAG with Orthogonal tRNA/Synthase Pair in JX33

To determine if the JX33 RF1-deletion permits an unnatural amino acid to be incorporated, we repeated the above experiments using the orthogonal tRNA_{CUA}^{Tyr}/LW1RS pair (to incorporate pActF at Uaa (unnatural amino acid). In JX2.0 cells, only the EGFP reporter containing a single TAG produced full-length EGFP protein. No full-length EGFP was detected in reporters containing 2-, 3-, or 6-TAGS by Western blot (FIG. 7D) or in-cell fluorescence (FIGS. 7E and 7F). LW1RS is less active than wt TyrRS in aminoacylating their respective amino acids, explaining why small amounts of EGFP were detected in the 2- and 3-TAG reporters in JX2.0 with tRNA_{CUA}^{Tyr}/TyrRS but not with tRNA_{CUA}^{Tyr}/LW1RS.

In JX3.0, however, large amounts of full-length EGFP were produced in the 1-, 2-, and 3-TAG reporters using the tRNA_{CUA}^{Tyr}/LW1RS pair (FIG. 7D). Protein yield did not diminish when the number of UAG codons increased from 1 to 3 (Table 2). In-cell fluorescence measurement confirmed that pActF was incorporated into EGFP at multiple sites in JX33 and not in JX2.0. In-cell fluorescence measurement showed a small decrease in fluorescence intensity when pActF was incorporated at the second UAG site but no further decrease at the third UAG site. The 6-TAG mutant also produced full-length protein in the JX33 strain (FIG. 7D), but with a lower yield than the 1-, 2-, or 3-TAG mutants and exhibited no green fluorescence (FIGS. 7E and 7F). The introduction of 6pActF into this protein likely affects its folding and stability, reducing protein yields and fluorescence.

TABLE 2
Protein yields of the various EGFP-TAG
constructs expressed in JX2.0 and JX33.

| Protein species | JX2.0 (mg/L) | JX33 (mg/L) |
|---|---|---|
| wild-type EGFP (no TAG) | | 14.9 |
| 6 - Tyr | N/D | 6.8 |
| 10 - Tyr | N/D | 0.4 |
| 1 - pActF | 1.8 | 3.5 |
| 2 - pActf | N/D | 3.5 |
| 3 - pActF | N/D | 5.4 |
| 6 - pActF | N/D | 0.5 |
| 10 - pActF | N/D | 0.5 |
| 3 - pAzdF | N/D | 0.8 |
| 3 - ActK | N/D | 1.0 |
| 3 - pCmF | N/D | 0.2 |
| 3 - pIodF | N/D | 0.9 |

For Table 2, protein yields were determined from samples purified first with Ni-NTA chromatography followed by FPLC using an anion exchange column. FPLC purification is necessary to remove truncated protein products when the His6 tag is appended at the N-terminus. N/D: not determined due to too low yield.

Yields for JX33 samples were 3.5-5.4 mg/L among the 1-, 2-, and 3-TAG mutants. These yields represent 24-36% of wild-type EGFP expressed without UAG codons, and are drastically higher than those (0.17% (Huang, et al., Mol. Biosyst. 6, 683-6 (2010)) and 0.86% (Kava, et al., Chembiochem 10, 2858-61 (2009)) for 3-TAG) reported previously. The yield for EGFP with pActF incorporated at 6 UAG sites was 0.5 mg/L. All yields were determined from proteins purified by Ni-NTA chromatography followed by more stringent anion exchange FPLC. Previously reported yields may have been inflated by truncated protein products, which are included in Ni-NTA purification of N-terminally tagged proteins.

Mass spectrometry was used to confirm incorporation of pActF at UAG sites. Fidelity for unnatural amino acid incorporation depends primarily on the substrate specificity of the orthogonal synthetase, and LW1RS specificity for pActF has been established. Consistently, electrospray ionization mass spectrometry (ESI-MS) of intact EGFP protein expressed by the 1-TAG reporter in JX33 showed two peaks (27801 and 27897 Da), corresponding to the mature pActF-containing EGFP minus the N-terminal methionine (theoretical mass 27799.2 Da) and the unfolded pActF-containing EGFP (theoretical mass 27899.2 Da), respectively. ESI-MS analysis of EGFP expressed by the 2- and 3-TAG reporters showed a single peak at 27898 and 27924 Da, respectively. These peaks lie within ±2 Da of the theoretical masses of the 2 and 3 pActF-containing mature EGFP minus the N-terminal methionine (27896.3 and 27922.3 Da, respectively). No peaks were observed in any sample corresponding to mutant EGFP containing any natural amino acid at the UAG position. This corroborates our Western blot and in-cell fluorescence data showing no significant EGFP expression in the absence of pActF (FIGS. 7D and 7E).

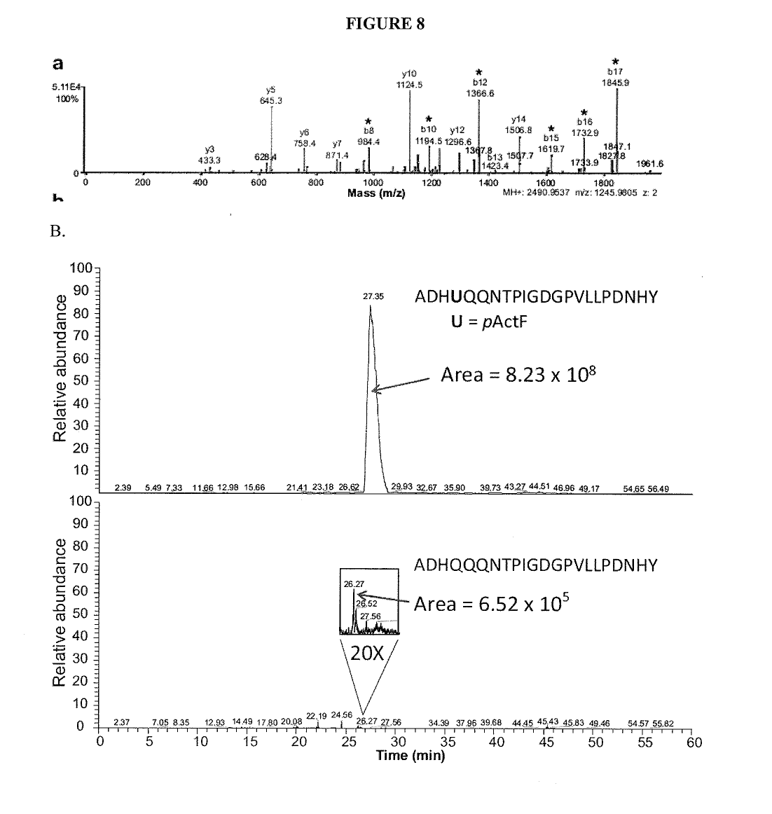

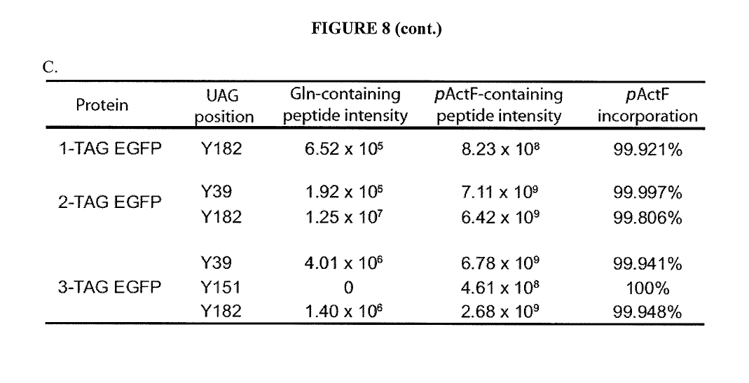

Liquid chromatography tandem mass spectrometry (LC-MS/MS) of chymotrypsin-digested protein samples was carried out to confirm the sequence of peptides containing UAG sites. LC-MS/MS allows characterization of mutant proteins with high sensitivity and dynamic range. The fragment ion masses were unambiguously assigned confirming the site-specific incorporation ofpActF at the UAG site for all 1-, 2-, and 3-TAG EGFP mutants (FIG. 8A). Extracted ion chromatograms (EIC) clearly showed that the peptide with pActF incorporated at the UAG site was the dominant species, with only trace amounts of Gln-containing peptide (FIGS. 8B and 8C). Because both pActF and Gln have neutral side chains, the two peptides containing pActF or Gln at the UAG site are expected to be similar in ionization efficiency (Chen, et al., J. Mol. Biol. 371, 112-22 (2007)). The peptide intensity calculated from peak area in EIC was therefore used to determine the incorporation fidelity of pActF at all UAG sites (FIGS. 8B and 8C). Incorporation of pActF in JX33 was found >99.81% at all UAG sites (FIG. 8C). Overall, the results demonstrate that, like JX1.0, the JX33 strain can efficiently and specifically incorporate the unnatural amino acid pActF into a protein at multiple sites encoded by UAG.

### I. Example 8

### Multiple UAG Sites can be Simultaneously Suppressed in JX33

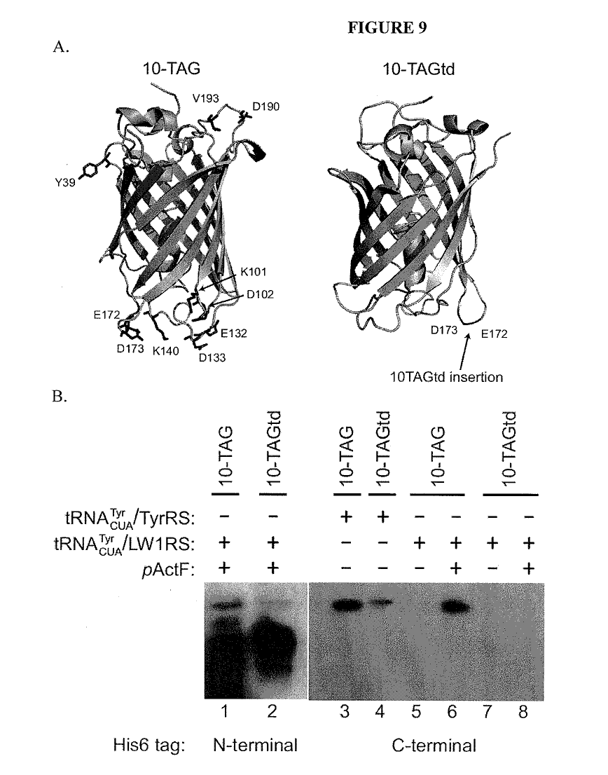

To assess whether multiple UAG sites can be suppressed (reassigned) simultaneously for amino acid incorporation in JX33, two 10-TAG containing EGFP reporters were prepared. One has 10 TAGs inserted across various loop regions in EGFP (10-TAG), and the other has 10 TAGs inserted in tandem in one loop (10-TAGtd, FIG. 9A). Loop sites were chosen to minimize potential mutational effects to protein folding and stability. Using the tRNA_{CUA}^{Tyr}/LW1RS to incorporate pActF into these mutants, full-length EGFP was produced, despite the appearance of truncation products (FIG. 9B). The expression of 10-TAGtd reporter was lower than the 10-TAG reporter, likely because 10 consecutive pActFs would have a greater affect on EGFP folding and stability.

To facilitate protein yield determination, the His6 tag was moved to the C-terminus of the 10 TAG reporters. Proteins were purified by Ni-NTA chromatography followed by FPLC. Incorporation of tyrosine using the tRNA_{CUA}^{Tyr}/TyrRS pair yielded full-length EGFP for both 10 TAG mutants, with a similar decrease in expression of the 10-TAGtd reporter (FIG. 9B). For the 10-TAG mutant, protein yields were 0.4 mg/L for tyrosine incorporation and 0.5 mg/L for pActF incorporation. Fluorescence was abolished in all 10-TAG reporters regardless of identity of the amino acid incorporated, suggesting that fluorescence was affected by these mutations. The expression level of the 10-TAG reporter was reduced in comparison to the 1-, 2-, and 3-TAG reporters, presumably due to folding and stability issues caused by the large number of mutations. Nonetheless, the ability to produce any level of protein with 10 unnatural amino acids selectively incorporated has never been reported, and the present results are novel. The RF1 deficient strains reported here thus allow for unlimited incorporation of unnatural amino acids, assuming that protein folding and stability are not overly affected.

### J. Example 9

### Multiple Unnatural Amino Acids can be Incorporated at Reassigned UAG Sites

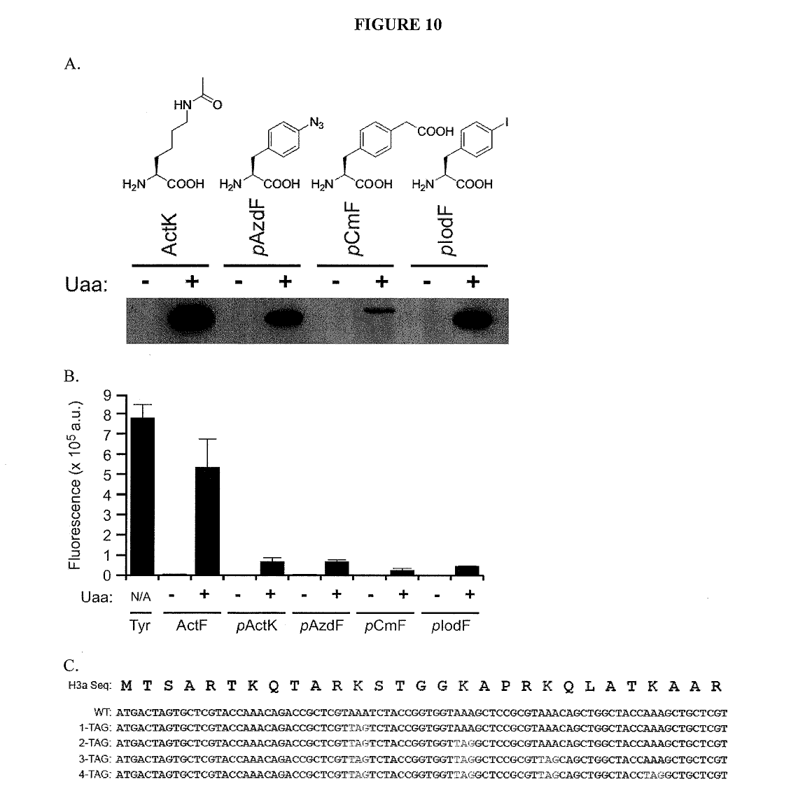

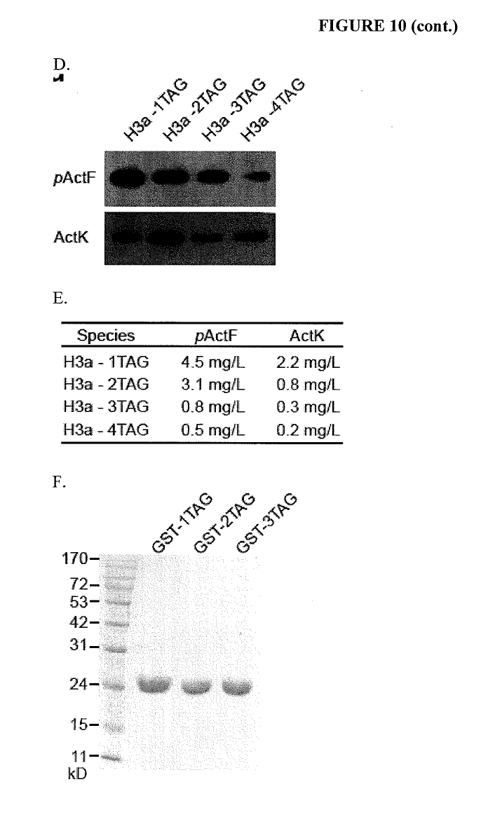

To determine if incorporation at multiple UAG sites in JX33 was generally applicable to different unnatural amino acids, the expression of the 3-TAG EGFP reporter with a variety of unnatural amino acids was detected. EGFP was efficiently expressed with N^{ε}-acetyl-L-lysine (ActK) (Neumann, et. al., Nat. Chem. Biol. 4, 232-4 (2008)), p-azido-L-phenylalanine (pAzdF), p-carboxymethyl-phenylalanine (pCmF) (Xie, et al., ACS Chem. Biol. 2, 474-8 (2007)), and p-iodo-phenylalanine (pIodF) (Xie, et al., Nat. Biotechnol. 22, 1297-1301 (2004)), as shown by Western blot (FIG. 10A) and in-cell fluorescence (FIG. 10B). No full-length EGFP was detected by Western blot in the absence of the unnatural amino acid in the growth media. Protein yields varied with the unnatural amino acid, and were reduced in comparison to pActF. These differences reflect the relative activity of the orthogonal synthetases; LW1RS (specific for pActF) was the most active among those tested. About 1 mg/L of purified protein containing ActK, pAzdF, and pIodF at 3 positions was obtained (Table 1). ActK was incorporated using an orthogonal tRNA/aaRS pair derived from the M. barkeri tRNA^{Pyl}/PylRS, and the other 3 Uaas were incorporated using orthogonal pairs derived from the M. jannaschii tRNA^{Tyr}/TyrRS, indicating that JX33 is compatible for usage with different orthogonal tRNA/aaRS pairs. The results show that UAG codons in JX33 can be used to encode different natural or unnatural amino acids at multiple sites.

To demonstrate the use of the JX33 strain in the expression of other proteins, human histone H3a was expressed in JX33 with ActK, with pActF incorporated at 1, 2, 3, or 4 UAG codons placed at known acetylation sites (FIG. 10C). In JX2.0 cells with more than 1 UAG site, no expression of H3a protein was detected. In JX33, all H3a mutants were successfully expressed in full length in the presence of pActF or ActK (FIG. 10D). Protein yields are shown in FIG. 10E.

pActF was also incorporated into glutathione S-transferase (GST) constructs at 1, 2, and 3 UAG sites in JX33 (FIG. 10F). The protein yields were 67 (±11), 57 (±12), and 68 (±9) mg/L for 1-, 2-, and 3-TAG mutants, respectively. Similar to EGFP, the GST expression yield did not decrease when the number of UAG sites increased from 1 to 3. Taken together, these results indicate that UAG codons in JX33 can be used to encode unnatural amino acids at multiple sites in different proteins.

### K. Example 10

### Effect of RF1 on TAG-Terminated Endogenous Genes

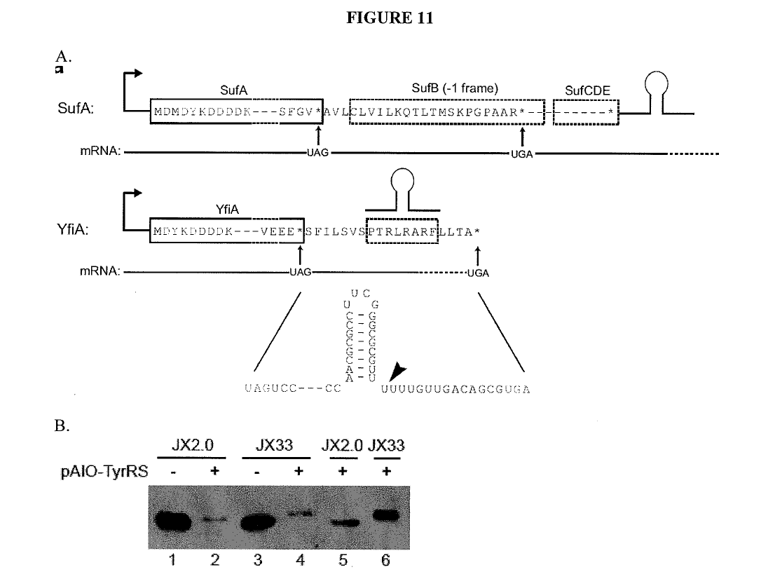

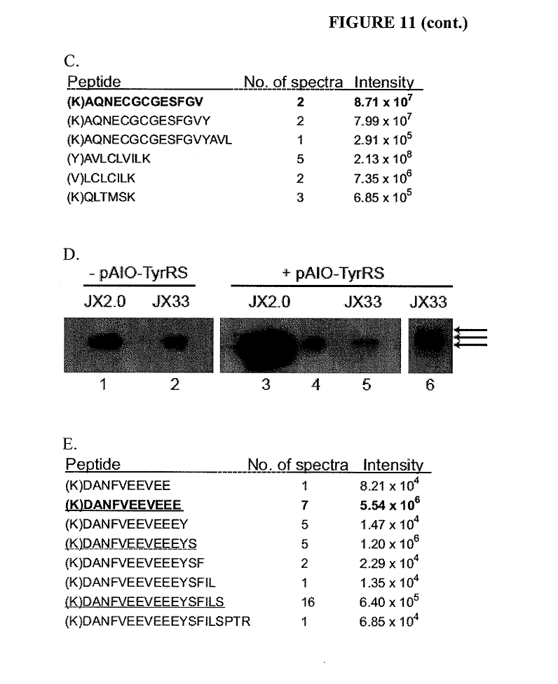

To determine the effect of RF1 disruption on the over 300 endogenous E. coli genes that are terminated with TAG, the genes were divided into two categories defined by their downstream context (FIG. 11A). The majority of the TAG-ending genes have a secondary in-frame stop codon (UAA or UGA) downstream in the mRNA transcript before a transcriptional terminator, as represented by sufA. Upon RF1 deletion and introduction of an amber suppressor tRNA/synthetase pair, translation of these genes is expected to extend to the next stop codon in JX33. To facilitate sensitive detection, a scarless insertion of a FLAG-tag was appended to the N-terminus of the sufA gene in the JX2.0 and JX33 strains. As expected, SufA protein purified from JX33 harboring pAIO-TyrRS showed an increase in size on a Western blot corresponding to the expected molecular weight increase from extension to the next stop codon (FIG. 11B).

In contrast, there was no detectable extension of SufA protein in JX2.0 harboring pAIO-TyrRS. Protein samples from JX33 harboring pAIO-TyrRS were analyzed by LC-MS/MS (FIG. 11C). Numerous peptide fragments were found that represent the extended peptide, confirming that translation was extended to the next stop codon and the UAG codon was suppressed with Tyr. The non-extended peptide fragment was also detected by mass spectrometry although it was not detectable on Western blot (FIG. 11C in bold).

The second category of genes has a transcriptional terminator between the UAG and the next in frame UAA or UGA, as represented by yfiA. Upon removal of RF1, the ribosome is expected to stall at the 3′ end of the mRNA as defined by the terminator hairpin. A scarless N-terminal FLAG tag was attached to the yfiA gene in JX2.0 and JX33. Western blotting of YfiA expressed in the presence of pAIO-TyrRS showed a dramatic reduction of protein expression as well as the appearance of multiple bands in JX33 in comparison to JX2.0 (FIG. 11D).

The tmRNA trans-translation mechanism likely explains the result (Moore and Sauer, Annu. Rev. Biochem. 76, 101-24 (2007)). Suppression of the UAG in JX33 results in the ribosome stalling at the end of the mRNA transcript. The stalled ribosome is recognized by tmRNA, which releases the ribosome and induces the degradation of the mRNA and extended polypeptide. In JX2.0, the presence of RF1 allows stoppage at the UAG codon to produce wild-type protein, and thus yfiA has much lower expression in JX33 than in JX2.0. To verify this, we analyzed YfiA purified from JX33 using LC-MS/MS to determine the identity of each band. C-terminally extended peptides that indicate extension to the next in-frame UGA stop codon were not observed by Western or MS. This is presumably because the mRNA is processed back to the terminator hairpin, making the distal poly-U portion of the hairpin (arrow in FIG. 11A) the 3′ end of the mRNA as previously reported (Abe, et al., Genes Cells 4, 87-97 (1999)). No proteins with the C-terminus extended to the end of mRNA were detected, indicating that these proteins were efficiently degraded via the tmRNA surveillance mechanism. The three bands resolved in lane 6 on the Western blot correspond to extensions of 0, 2, or 6 amino acids from the UAG site, respectively, with 0 and 2 amino acid extension being the dominant species in the Western blot (FIG. 11D, 40, 44, and 16% of total intensity for 0, 2, and 6 amino acid extension, respectively).

Lastly, protein expression of SufA and YfiA in the absence of pAIO-TyrRS was similar in both strains, most likely due to the weak termination ability of mutant RF2 for the UAG codon.

### L. Example 11

### JX33 Includes a Novel A293E Mutation

To further characterize JX3.0 (slow growing JX31 and fast growing JX33), full genomic sequencing was performed on JX2.0, JX3.0 (JX31 and JX33), and compared to that of E. coli K-12 MG1655. Both JX2.0 and JX3.0 showed gene deletions identical to the parental MDS42 strain, a multiple-deletion derivative of MG1655 (Posfai et al., Science 312, 1044-6 (2006)). The knockout of prfA by the Cm^{R} cassette in JX3.0 was confirmed, and this is the only deletion difference between JX2.0 and JX3.0. No other differences and mutations were found between JX2.0, JX31 and MG 1655. These results show that RF1 can be knocked out from JX2.0 without incurring compensatory mutations in other genes, indicating that RF1 is nonessential in JX2.0.

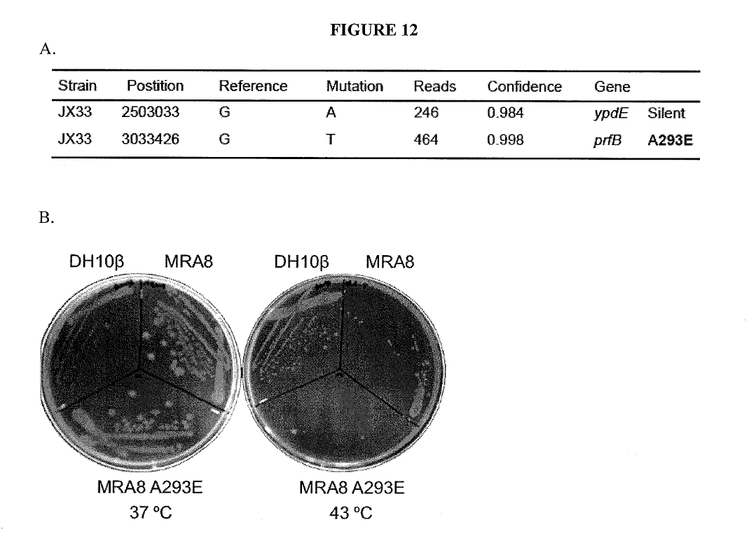

Two single nucleotide polymorphisms (SNPs) were found between JX2.0 and JX33 (FIG. 12A). One is a silent mutation in the coding region of the gene ypdE and the other results in an amino acid change (A293E) in RF2. No other mutations were found in JX2.0 and JX33.

The A293E mutation has not been discovered in any previous complementation screens for RF1 deficiency (Ito, et al., Proc. Natl. Acad. Sci. U.S.A. 95, 8165-9 (1998); Zhang, et al., J. Mol. Biol. 242, 614-8 (1994); Dahlgren and Ryden-Aulin, Biochimie 82, 683-91 (2000); Kaczanowska and Ryden-Aulin, J. Bacteriol. 186, 3046-55 (2004)). To characterize this novel mutation, we first determined if it was necessary for the survival of JX33.

To determine if the A293E mutation in RF2 could rescue RF1 function in E. coli, the endogenous RF2 gene of a temperature sensitive RF1 (tsRF1) strain (MRA8) was replaced with the RF2(A293E) gene from JX33 to create MRA8 A293E (Datsenko, et al., Proc. Natl. Acad. Sci. U.S.A. 97, 6640-5 (2000)). If this mutation was sufficient to confer survival without RF1, it should complement the tsRF1 deficiency and rescue growth of this strain at 43° C. No difference in growth phenotype was observed between the parental (MRA8) and mutant (MRA8 A293E) strains (FIG. 12B), indicating that the A293E mutation is not able to rescue RF1 temperature sensitive phenotype in E. coli.

### M. Example 12

### JX33 Allows for Efficient Incorporation of Non-Naturally Occurring Amino Acids in Multiple Proteins

JX33 is a novel RF1 knockout strain with unique properties for incorporation non-naturally occurring amino acids (e.g., mutant, natural amino acids (non-native), or unnatural (non-naturally occurring) amino acids) without reduced efficiency. JX33 is stable, autonomous and has no major growth or other deleterious defects. The present results show that, surprisingly, at least 10 amino acids could be incorporated at TAG sites in JX33.

Reduced yield was observed with additional UAGs in H3a, likely because all four UAG sites in H3a are close to each other. Mutant synthetases evolved for unnatural amino acids are not as active as the wild type synthetases, and thus generate less aminoacylated orthogonal tRNAs. Binding of natural aminoacyl-tRNAs to elongation factors and the ribosome has been evolutionary tuned for optimal decoding, while the orthogonal tRNA and unnatural amino acid have not been fully optimized. Moreover, standard tRNAs are subjected to post-transcriptional modification for specific and efficient decoding of cognate codons. The four UAG codons lying within a 15 amino acid stretch in the N-terminus of the H3a protein form a cluster of “rare” codons, resulting in reduced protein expression (Kane (1995) Curr. Opin. Biotechnol. 6:494).

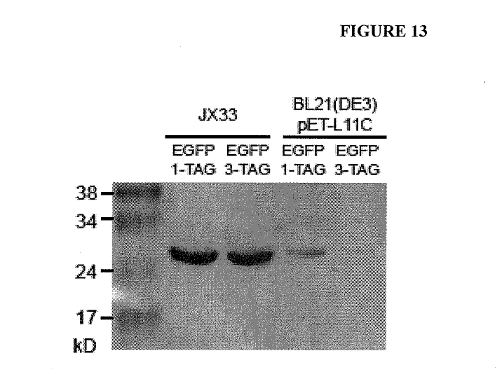

Overexpression of the C-terminus of ribosomal protein L11 (L11C) enables the incorporation of unnatural amino acids at TAG sites, though at reduced efficiency (Huang et al. (2010) Mol. Biosyst. 6; 683). For a side-by-side comparison with the earlier report, pActF was incorporated into EGFP at identical 1-TAG and 3-TAG sites using the Huang method and the present method, respectively (FIG. 13). The JX33 strain afforded 27 mg/L of EGFP for 1-TAG mutant and 23 mg/L for 3-TAG mutant; while coexpression of the L11C in BL21(DE3) afforded 4.6 mg/L for 1-TAG mutant and 1.2 mg/L for 3-TAG mutant. In comparison to the previous method, the present approach provides 5.9-fold protein for the 1-TAG mutant and 19-fold for the 3-TAG mutant, a marked increase for 1 UAG site and a more dramatic increase at 3 UAG sites. While no incorporation of unnatural amino acid at more than 3 UAG sites was achieved the previous method, JX33 can incorporate unnatural amino acid at 6 to 10 UAG sites.

Non-stop incorporation of pActF into EGFP was also observed in the slow growing JX3.0 strain, JX31. The protein yields for 1-, 2- and 3-TAG EGFP mutants from JX31 were 5.7 (±0.4), 6.1 (±0.5) and 7.0 (±0.5) mg/L, respectively. This result suggests that the RF2(A293E) mutation is not required for efficient incorporation of Uaa at multiple TAG sites.

JX33 is capable of incorporation of multiple unnatural amino acids at multiple sites. Selective incorporation of unnatural amino acids at multiple sites opens new possibilities in protein research and laboratory evolution. For instance, multiple heavy-atom containing unnatural amino acids (e.g., pIodF) allows for phase determination of proteins with large molecular weight. Multiple chemical handles (e.g., pActF) allows for selective PEGylation or glycosylation at multiple sites. Multiple fluorescent unnatural amino acids can facilitate single molecule imaging. Multi-site posttranslational modification mimics (e.g., ActK and pCmF) can be used to study epigenetics and signal transduction pathways.

### Informal Sequence Listing

The DNA sequence encoding release factor 2 (RF2) in E. coli str. K-12 substr. MG1655 (NCBI locus NC_{—}000913) follows:

| (SEQ ID NO: 1) | |
|---|---|
| 1 | atgtttgaaa ttaatccggt aaataatcgc attcaggacc tcacggaacg ctccgacgtt |
| 61 | cttagggggt atctttgact acgacgccaa gaaagagcgt ctggaagaag taaacgccga |
| 121 | gctggaacag ccggatgtct ggaacgaacc cgaacgcgca caggcgctgg gtaaagagcg |
| 181 | ttcctccctc gaagccgttg tcgacaccct cgaccaaatg aaacaggggc tggaagatgt |
| 241 | ttctggtctg ctggaactgg ctgtagaagc tgacgacgaa gaaaccttta acgaagccgt |
| 301 | tgctgaactc gacgccctgg aagaaaaact ggcgcagctt gagttccgcc gtatgttctc |
| 361 | tggcgaatat gacagcgccg actgctacct cgatattcag gcggggtctg gcggtacgga |
| 421 | agcacaggac tgggcgagca tgcttgagcg tatgtatctg cgctgggcag aatcgcgtgg |
| 481 | tttcaaaact gaaatcatcg aagagtcgga aggtgaagtg gcgggtatta aatccgtgac |
| 541 | gatcaaaatc tccggcgatt acgcttacgg ctggctgcgt acagaaaccg gcgttcaccg |
| 601 | cctggtgcgt aaaagcccgt ttgactccgg cggtcgtcgc cacacgtcgt tcagctccgc |
| 661 | gtttgtttat ccggaagttg atgatgatat tgatatcgaa atcaacccgg cggatctgcg |
| 721 | cattgacgtt tatcgcacgt ccggcgcggg cggtcagcac gttaaccgta ccgaatctgc |
| 781 | ggtgcgtatt acccacatcc cgaccgggat cgtgacccag tgccagaacg accgttccca |
| 841 | gcacaagaac aaagatcagg ccatgaagca gatgaaagcg aagctttatg aactggagat |
| 901 | gcagaagaaa aatgccgaga aacaggcgat ggaagataac aaatccgaca tcggctgggg |
| 961 | cagccagatt cgttcttatg tccttgatga ctcccgcatt aaagatctgc gcaccggggt |
| 1021 | agaaacccgc aacacgcagg ccgtgctgga cggcagcctg gatcaattta tcgaagcaag |
| 1081 | tttgaaagca gggttatga. |

The RF2 protein encoded by SEQ ID NO:1 (NCBI locus NC_{—}000913) follows. An in-frame premature UGA termination codon is located within the prfB sequence, and a naturally occurring +1 frameshift can be used for synthesis of RF2. The Thr at position 246 can be changed to an Ala for improved recognition of the UAA stop codon.

(SEQ ID NO: 2)
MFEINPVNNRIQDLTERSDVLRGYLDYDAKKERLEEVNAELEQPDVWNEP
ERAQALGKERSSLEAVVDTLDQMKQGLEDVSGLLELAVEADDEETFNEAV
AELDALEEKLAQLEFRRMFSGEYDSADCYLDIQAGSGGTEAQDWASMLER
MYLRWAESRGFKTEIIEESEGEVAGIKSVTIKISGDYAYGWLRTETGVHR
LVRKSPFDSGGRRHTSFSSAFVYPEVDDDIDIEINPADLRIDVYRTSGAG
GQHVNRTESAVRITHIPTGIVTQCQNDRSQHKNKDQAMKQMKAKLYELEM
QKKNAEKQAMEDNKSDIGWGSQIRSYVLDDSRIKDLRTGVETRNTQAVLD
GSLDQFIEASLKAGL.
(SEQ ID NO: 3)
ADHUQQNTPIGDGPVLLPDNHY (U = pActF or Q).
(SEQ ID NO: 4)
DYKDDDDK.
(SEQ ID NO: 5)
HHHHHH.
(SEQ ID NO: 6)
cuuagggggu aucuuugacu acgac.
(SEQ ID NO: 7)
LRGYLDYD.
(SEQ ID NO: 8)
cuccgcggcu aucuugacua cgac.
(SEQ ID NO: 9)
MDMDYKDDDDK.
(SEQ ID NO: 10)
SFGV.
(SEQ ID NO: 11)
AVLCLVILKQTLTMSKPGPAAR.
(SEQ ID NO: 12)
VEEE.
(SEQ ID NO: 13)
SFILVSVPTRLRARFLLTA.
(SEQ ID NO: 14)
ccaacgcgcc uucgggcgcg uuuuuuguug acagcguga.
(SEQ ID NO: 15)
KAQNECGCGESFGV.
(SEQ ID NO: 16)
KAQNECGCGESFGVY.
(SEQ ID NO: 17)
KQQNECGCGESFGVYAVL.
(SEQ ID NO: 18)
YAVLCLVILK.
(SEQ ID NO: 19)
VLCLCILK.
(SEQ ID NO: 20)
KQLTMSK.
(SEQ ID NO: 21)
KDANFVEEVEE.
(SEQ ID NO: 22)
KDANFVEEVEEE.
(SEQ ID NO: 23)
KDANFVEEVEEEY.
(SEQ ID NO: 24)
KDANFVEEVEEEYS.
(SEQ ID NO: 25)
KDANFVEEVEEEYSF.
(SEQ ID NO: 26)
KDANFVEEVEEEYSFIL.
(SEQ ID NO: 27)
KDANFVEEVEEEYSFILS.
(SEQ ID NO: 28)
KDANFVEEVEEEYSFILSPTR

1. A viable, recombinant, release factor 1 (RF1)-deficient bacterial cell.

2. (canceled)

3. The cell of claim 2, wherein the bacterial cell is Escherichia coli from a parental strain selected from the group consisting of REL606, BL21, BL21 (DE3), and DH10βf.

4. The cell of claim 1, wherein the cell comprises functional release factor 2 (RF2).

5. The cell of claim 4, wherein the RF2 comprises an alanine at the amino acid position corresponding to 246 of SEQ ID NO:2 and/or a glutamic acid at the amino acid position corresponding to 293 of SEQ ID NO:2.

6. (canceled)

7. The cell of claim 1, wherein the UAG codon is recognized by an aminoacylated tRNA, and results in incorporation of an amino acid into a nascent protein strand.

8. The cell of claim 7, wherein the amino acid is a non-naturally occurring amino acid.

9. The cell of claim 7, wherein the amino acid is selected from the group consisting of tyrosine, glutamine, and tryptophan.

10. The cell of claim 1, wherein the cell grows at the same rate or within 10% of the rate of a wild type bacterial cell.

11. The cell of claim 1, wherein the cell comprises
(i) a first exogenous recombinant nucleic acid encoding a protein comprising a mutant amino acid, wherein said mutant amino acid is encoded by a TAG codon where said first exogenous recombinant nucleic acid is DNA, or by a UAG codon where said first exogenous recombinant nucleic acid is RNA;
(ii) a second exogenous recombinant nucleic acid encoding an orthogonal tRNA comprising a CUA anticodon; and
(iii) a third exogenous recombinant nucleic acid encoding an orthogonal synthetase capable of functionally binding to said orthogonal tRNA.

12-13. (canceled)

14. A method for reassigning the UAG codon in a bacterial cell, comprising rendering the bacterial cell release factor 1 (RF1) deficient.

15. (canceled)

16. The method of claim 15, wherein the bacterial cell is Escherichia coli from a parental strain selected from the group consisting of REL606, BL21, BL21 (DE3), and DH10βf.

17. The method of claim 14, wherein said rendering comprises recombinant disruption of the endogenous RF1 gene in the bacterial cell.

18. The method of claim 14, wherein the wherein the cell comprises functional release factor 2 (RF2).

19. The method of claim 18, wherein the RF2 comprises an alanine at the amino acid position corresponding to 246 of SEQ ID NO:2 and/or a glutamic acid at the amino acid position corresponding to 293 of SEQ ID NO:2.

20. (canceled)

21. The method of claim 14, wherein the UAG codon is recognized by an aminoacylated tRNA, and results in incorporation of an amino acid into a nascent protein strand.

22. The method of claim 21, wherein the amino acid is a non-naturally occurring amino acid.

23. The method of claim 21, wherein the amino acid is elected from the group consisting of tyrosine, glutamine, and tryptophan.

24. A method of producing a protein comprising a mutant amino acid in a bacterial cell comprising:
(i) transfecting the cell of claim 1 with:
(a) a first exogenous recombinant nucleic acid encoding a protein comprising a mutant amino acid, wherein said mutant amino acid is encoded by a TAG codon where said first exogenous recombinant nucleic acid is DNA, or by a UAG codon where said first exogenous recombinant nucleic acid is RNA;
(b) a second exogenous recombinant nucleic acid encoding an orthogonal tRNA comprising a CUA anticodon; and
(c) a third exogenous recombinant nucleic acid encoding an orthogonal synthetase capable of functionally binding to said orthogonal tRNA;
(ii) allowing the cell to express the protein, thereby producing the protein comprising a mutant amino acid.

25. The method of claim 24, wherein the mutant amino acid is a naturally occurring amino acid.

26. The method of claim 24, wherein the mutant amino acid is a non-naturally occurring amino acid.

27-33. (canceled)
